(19) United States
(12) Patent Application Publication
(10) Pub. No.: US 2025/0009451 A1
(43) Pub. Date: Jan. 9, 2025

(54) IMAGE GUIDED ROBOTIC SPINE INJECTION SYSTEM

(71) Applicant: The Johns Hopkins University, Baltimore, MD (US)

(72) Inventors: Henry PHALEN, Baltimore, MD (US); Cong GAO, Baltimore, MD (US); Adam MARGALIT, Baltimore, MD (US); Amit JAIN, Baltimore, MD (US); Mehran ARMAND, Baltimore, MD (US); Russell H. TAYLOR, Baltimore, MD (US)

(73) Assignee: The Johns Hopkins University, Baltimore, MD (US)

(21) Appl. No.: 18/711,344

(22) PCT Filed: Dec. 5, 2022
(86) PCT No.: PCT/US2022/051841

§ 371 Date: May 17, 2024

Related U.S. Application Data
(60) Provisional application No. 63/286,376, filed on Dec. 6, 2021.

(51) Int. Cl. A61B 34/32; A61B 34/10; A61B 34/20; A61B 90/00

(52) U.S. Cl. CPC A61B 34/32; A61B 34/10; A61B 34/20; A61B 90/36; A61B 2034/105; A61B 2034/107

(57) ABSTRACT

An image-guided robotic spine injection system includes an injection robot registered to an interoperative imaging system for real-time guidance. The system includes a guidance system to communicate with said injection robot and said interoperative imaging system during an injection procedure. The guidance system includes a preoperative injection plan based on preoperative imaging data of a subject's spine, and includes anatomical features identified as preoperative registration markers. The guidance system receives interoperative imaging data from said interoperative imaging system of said subject's spine. The guidance system receives an indication of anatomical features identified as interoperative registration markers that correspond in a one-to-one relationship to each of said preoperative registration markers. The guidance system registers said interoperative registration markers with said preoperative registration markers to transform said preoperative injection plan to an interoperative injection plan. The guidance system provides instructions to said injection robot to perform autonomous injections into said subject's spine.

### CROSS-REFERENCE OF RELATED APPLICATION

This application claims priority to U.S. Provisional Application No. 63/286,376, filed Dec. 6, 2021, which is incorporated herein by reference in its entirety.

### STATEMENT REGARDING FEDERALLY SPONSORED RESEARCH OR DEVELOPMENT

This invention was made with government support under grant EB023939 awarded by the National Institutes of Health, and grant DGE-1746891 by the National Science Foundation. The government has certain rights in the invention.

### BACKGROUND

### 1. Technical Field

The currently claimed embodiments of the present invention relate to spine injection, and more specifically to systems and methods for image-guided robotic spine injection.

### 2. Discussion of Related Art

Epidural steroid injections are a cornerstone of conservative treatment of a variety of cervical and lumbar spinal diseases including stenosis, radiculopathy, and pain. These procedures have been performed since the 1950s and are the most frequently performed procedure in pain medicine in the United States. When administered appropriately, they can be very effective in treating pain, restoring function, and avoiding surgery.

Transforaminal epidural steroid injection in the lumbar spine is a common non-surgical treatment for lower back pain or sciatica. Globally, between 60-80% of people are estimated to experience lower back pain in their lifetime and it is among the top causes of adult disability [1], [2]. Efficacy of treatment is reported as 84%, with adequate targeting of the injection site thought to be critical to successful treatment [3]. There is wide variability in the literature, ranging from 0-100%, regarding the efficacy of lumbar epidural injections for pain control.^{4,6,7} However, the most highly cited prospective randomized control trial demonstrated an efficacy of 84% (defined as pain reduction greater than 50% 1 year after treatment).⁷ Factors associated with variable success may include spinal instability, chronicity and grade of nerve root compression, and procedure technique and needle tip accuracy.^{4,7,8}

Epidural injection in the lumbar spine is typically performed by a clinician using fluoroscopy. The clinician will acquire several images before and during manual insertion of the needle. When satisfied with needle placement, the clinician will inject a steroid and remove the needle. Several injections at different levels of the spine may be performed in sequence. Given the importance of accurate targeting and the proximity to critical anatomy, robotic systems have been considered as a tool to perform these injections. Various imaging technologies have been used for guidance of these systems including MRI [4], [5], [6], [7], ultrasound [8], [9], and cone-beam CT [10]. However, MRI and CT machines are expensive, and are not commonly available in the orthopedic operating rooms. Furthermore, these 3D imaging modalities-MRI in particular—can greatly prolong the surgical procedure. Ultrasound data are often noisy, and it can be complicated to extract contextual information. Thus, ultrasound-guided needle injection requires longer scanning time and is limited in reconstruction accuracy [8]. Often, additional sensing modalities are needed along with ultrasound, such as force sensing [9].

In contrast, fluoroscopic imaging is fast and low-cost. In particular, C-arm X-ray machines are widely used in orthopedic operating rooms. X-ray imaging presents deep-seated anatomical structures with high resolution. A general disadvantage of fluoroscopy is that it adds to the radiation exposure of the patient and surgeon. However, orthopaedic surgeons use fluoroscopy for verification to gain “direct” visualization of the anatomy. As such, the use of fluoroscopy for navigation is intended to replace its use for manual verification images, resulting in similar radiation exposure compared to a conventional procedure. Fluoroscopic guided needle placement has been studied [11], [12], [13]. These approaches either require custom-designed markers to calibrate the robot end effector to the patient anatomy, or the surgeon's supervision to verify the needle placement accuracy.

Fiducial-free navigation uses purely image information to close the registration loop, and has been investigated in other robot-assisted orthopedic applications [14], [15]. Poses of the bone anatomy relative to the surgical tool have been estimated using image-based 2D/3D registration. For example, a mean positional error of 2.86±0.80 mm has been reported in cadaveric studies [15], which shows feasibility for orthopedic applications.

The 3 main approaches for administering epidural steroid injections in the lumbar spine include transforaminal, interlaminar, and caudal approaches.⁴ The main advantage of the transforaminal approach is the presumed ability to deliver medications as close as possible to the lumbar nerve roots.⁴ These injections are frequently delivered under fluoroscopic, computed tomography (CT), or ultrasound guidance in order to increase the accuracy of needle placement. There are few differences in outcomes between these modalities, although fluoroscopy is more commonly utilized.^{4,5} However, the effectiveness depends on accuracy of placement, and can vary based on the provider's experience and technique.

Furthermore, although rare, epidural injection cases have been linked to spinal cord or neural injuries.⁴ These cases are thought to be due to inadvertent vascular injection (up to 23% of cases) of corticosteroids,^{9,10} and unintentional intra-arterial injection of the steroid into a radiculomedullary artery that supplies the spinal cord, with resultant RBC agglutination and occlusion of the anterior spinal artery leading to cord infarction or direct vascular trauma or vasospasm leading to distal ischemic insult.

Improving accuracy and precision of injections would result in improvements in clinical benefit. Therefore, there remains a need for improved systems and methods for spine injections.

### SUMMARY

An embodiment of the present invention is an image-guided robotic spine injection system, including a spine injection robot having an end effector configured to hold an injection device, said spine injection robot being configured to be registered to an interoperative imaging system for real-time guidance of the injection device. The system further includes a guidance system configured to communicate with said spine injection robot and the interoperative imaging system during an injection procedure. The guidance system includes a preoperative injection plan for a planned injection procedure on a subject, the preoperative injection plan being based on preoperative imaging data of at least a portion of the subject's spine, the preoperative injection plan including multiple anatomical features identified as corresponding preoperative registration markers. The guidance system is configured to receive interoperative imaging data from the interoperative imaging system of at least the portion of the subject's spine. The guidance system is further configured to receive as input from a user an indication of multiple anatomical features identified as interoperative registration markers that correspond in a one-to-one relationship to each respective one of the preoperative registration markers. The guidance system is further configured to register the interoperative registration markers with the preoperative registration markers to transform the preoperative injection plan to an interoperative injection plan, and further configured to provide injection guidance instructions to the spine injection robot to perform autonomous injections into the spine of a subject by the injection device.

Another embodiment of the present invention is a method for image guidance for robotic spine injection. The method includes registering a spine injection robot to an interoperative imaging system for real-time guidance of an injection device coupled to the spine injection robot, receiving preoperative imaging data of a subject's spine, and generating, based on the preoperative imaging data, a preoperative injection plan for a planned injection procedure on the subject. The preoperative injection plan includes multiple anatomical features identified as corresponding preoperative registration markers. The method further includes receiving an indication of multiple anatomical features identified as interoperative registration markers that correspond in a one-to-one relationship to each respective one of the preoperative registration markers. The method further includes registering the interoperative registration markers with the preoperative registration markers to transform the preoperative injection plan to an interoperative injection plan, and providing injection guidance instructions to the spine injection robot to perform autonomous injections into the subject's spine by the injection device.

Another embodiment of the present invention is a non-transitory computer-readable medium storing a set of instructions for image-guided robotic spine injection, which when executed by a processor, configure the processor to register a spine injection robot to an interoperative imaging system for real-time guidance of an injection device coupled to the spine injection robot. The instructions further configure the processor to receive preoperative imaging data of a subject's spine and generate, based on the preoperative imaging data, a preoperative injection plan for a planned injection procedure on the subject. The preoperative injection plan includes multiple anatomical features identified as corresponding preoperative registration markers. The instructions further configure the processor to receive an indication of multiple anatomical features identified as interoperative registration markers that correspond in a one-to-one relationship to each respective one of the preoperative registration markers. The instructions further configure the processor to register the interoperative registration markers with the preoperative registration markers to transform the preoperative injection plan to an interoperative injection plan, and provide injection guidance instructions to the spine injection robot to perform autonomous injections into the subject's spine by the injection device.

### BRIEF DESCRIPTION OF THE DRAWINGS

Further objectives and advantages will become apparent from a consideration of the description, drawings, and examples.

FIG. 1 shows a picture of an image-guided robotic injection system of some embodiments.

FIG. 2 shows a fluoroscopy-guided robotic injection system of some embodiments.

FIG. 3 shows a calibration scheme used for a robotic injection system of some embodiments.

FIG. 4A shows an illustration of collecting multi-view C-arm images for the robotic injection system of some embodiments.

FIG. 4B shows a process for a multi-view registration workflow performed by the robotic injection system of some embodiments.

FIG. 5 presents an example of a simulated spine deformation in some embodiments.

FIG. 6 shows normalized 2D histograms of registration pose error reported in joint magnitudes of translation and rotation, for the robotic injection system of some embodiments.

FIG. 7 shows an example of robotic injection using the robotic system of some embodiments, on a cadaver specimen.

FIG. 8 shows a CT reconstruction of a radiopaque sawbones lumbar spine model used in some embodiments.

FIG. 9 shows an example of preoperative software modeling in some embodiments.

FIG. 10 shows freehand needle placement according to some embodiments.

FIG. 11A shows a robotic injection system of some embodiments.

FIG. 11B shows an image of a robotic arm with an attached injection device for the robotic injection system of FIG. 11A.

FIG. 12 shows a scatter plot demonstrating differences in precision of the needle tip in mm between the postoperative freehand fluoroscopic and robotic technique of some embodiments.

FIG. 13 shows differences from planned trajectories according to some embodiments.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed, without departing from the broad concepts of the current invention.

All references cited anywhere in this specification, including the Background and Detailed Description sections, are incorporated by reference as if each had been individually incorporated.

Some embodiments of the current invention relate to systems and methods for the administration of spinal injections using an image-guided autonomous robotic system, including but not limited to epidural steroid injections, transforaminal, interlaminar, and caudal injections, selective nerve root blocks, medial branch blocks, and radio frequency ablations. Some embodiments of the current invention may be used remotely and enable tele-surgery.

Some embodiments include obtaining preoperative imaging (including but not limited to computed tomography (CT) or magnetic resonance imaging (MRI) scans), intraoperative imaging (e.g., using a C-arm or an O-arm), and a robotic arm with an end effector designed to administer injections.

In some embodiments of the invention, spinal preoperative images may be digitally segmented. Intraoperative imaging of the spine may be obtained in multiple viewpoints, and landmark targets may be planned and annotated on a computerized platform. A software algorithm may be used to produce a fiducial-free 2D/3D registration plan according to some embodiments of the current invention. The robotic arm can be instructed to precisely orient the injector end effector toward the programmed target, and the preop plan can be executed with the robotic end effector under image guidance.

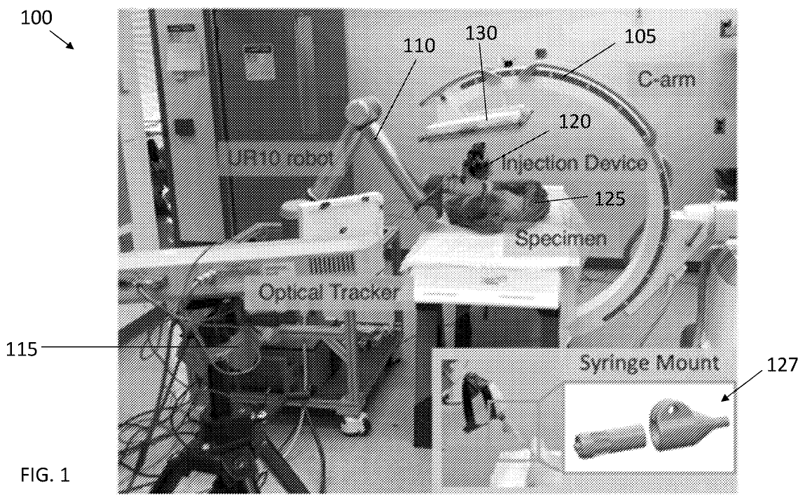

Image-guided robotic spine injection systems according to embodiments of the current invention can be seen in FIGS. 1-4 in the Examples below.

FIG. 1 shows a picture of an image-guided robotic injection system 100 of some embodiments. The robotic injection system 100 includes a C-arm 105, a spine injection robot 110 having an arm, a tracking system 115, and an injection device 120, for performing a procedure on a subject's spine 125. In the bottom right, a picture of a syringe mount 127 is shown. The injection device 120 attaches to the arm of the spine injection robot 110 by an end effector (not shown) in some embodiments.

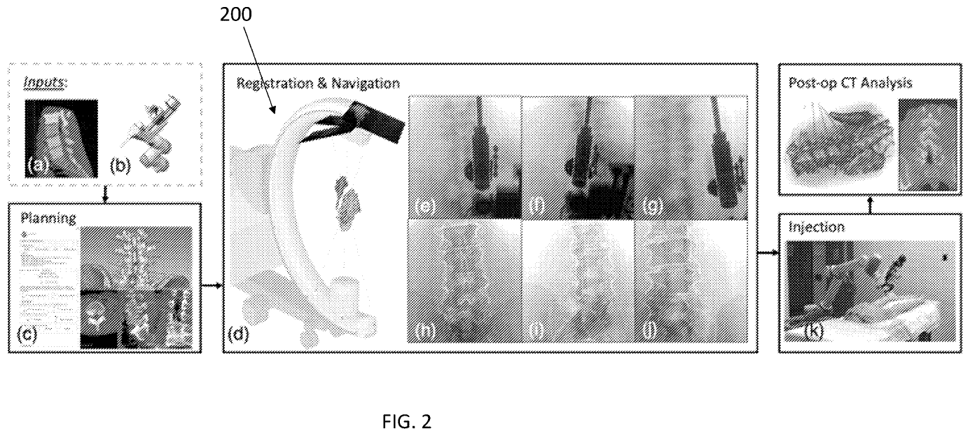

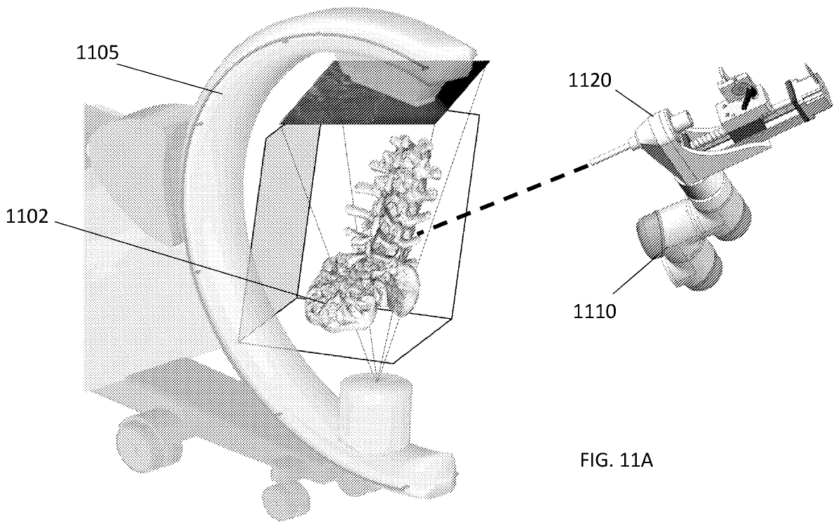

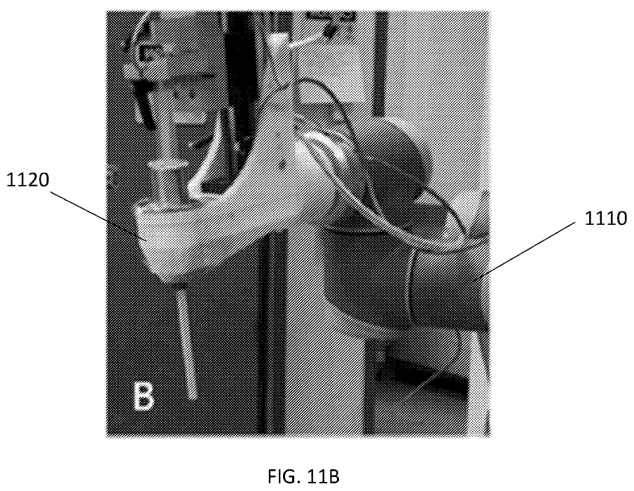

The robotic injection system 100 is similar to the embodiments of the robotic injection system 200, 1100 discussed with respect to FIG. 2 and FIG. 11A, and like reference numerals have been used to refer to the same or similar components. A detailed description of these components will be omitted, and the following discussion focuses on the differences between these embodiments. Any of the various features discussed with any one of the embodiments discussed herein may also apply to and be used with any other embodiments.

In the example of FIG. 1, the subject is a cadaveric specimen, however during clinical operation the subject would be a living patient. For example, in some embodiments of the robotic injection system 100, the robotic arm may be a UR-10 robotic arm (Universal Robots, Odense, Denmark), and the tracking system 115 may be an optical tracker.

Some embodiments of the robotic injection system 100 may be used for transforaminal spine injection under fluoroscopic guidance, that autonomously places needles for injection on the subject's spine 125 using only 2D fluoroscopic images for registration. The robotic injection system 100 may further include (as described below in FIG. 2) a planning module, a rigid-link robot platform that has a needle injection device, and a navigation pipeline that uses multi-view X-ray registration to automatically position the needle and perform injections.

The image-guided robotic injection system 100 includes a spine injection robot 110 comprising an end effector configured to hold an injection device 120, the spine injection robot 110 being configured to be registered to an interoperative imaging system 130 (e.g., mounted on C-arm 105) for real-time guidance of the injection device; and a guidance system (not shown) configured to communicate with the spine injection robot 110 and the interoperative imaging system 130 during an injection procedure. The guidance system may include a preoperative injection plan for a planned injection procedure on the subject's spine 125, the preoperative injection plan being based on preoperative imaging data of at least a portion of the subject's spine 125, the preoperative injection plan including a plurality of anatomical features identified as a corresponding plurality of preoperative registration markers. The guidance system 115 may be configured to receive interoperative imaging data from the interoperative imaging system 130 of at least the portion of the subject's spine 125. The guidance system may be further configured to receive as input from a user an indication of a plurality of anatomical features identified as a plurality of interoperative registration markers that correspond in a one-to-one relationship to each respective one of the plurality of preoperative registration markers. The guidance system may be further configured to register the plurality of interoperative registration markers with the preoperative registration markers to transform the preoperative injection plan to an interoperative injection plan, and the guidance system may be further configured to provide injection guidance instructions to the spine injection robot 110 to perform autonomous injections into the subject's spine 125 by the injection device 120.

In some embodiments, the plurality of anatomical features are at least a portion of each of a plurality of vertebrae of the subject's spine 125, and the registering the plurality of interoperative registration markers with the preoperative registration markers accounts for relative movement of vertebrae in the subject's spine 125 in the interoperative images compared to the preoperative images.

In some embodiments, the preoperative injection plan includes boundaries to prevent the injection device 120 from damaging the subject's spinal cord or other nerves.

In some embodiments, the robotic injection system 100 further includes a tracking system 115 configured to communicate with the guidance system. In this embodiment, the tracking system 115 is arranged to be registered to and track the spine injection robot 110, the end effector of the spine injection robot, a needle and injection device 120 when attached to the end effector, an imaging portion of the interoperative imaging system 130, and the plurality of vertebrae of the subject's spine 125 while in operation.

In some embodiments, the tracking system 115 provides closed-loop control of the spine injection robot 110 based on tracking information from the tracking system 115.

In some embodiments, the robotic injection system 100 further includes a preoperative planning module configured to receive preoperative imaging data of the at least the portion of the subject's spine 125, wherein the preoperative planning module is further configured to receive a planned injection point and a planned destination point from a user and to display a corresponding calculated needle path to the user.

In some embodiments, the robotic injection system 100 further includes the interoperative imaging system 130. In some embodiments, the preoperative imaging data is three-dimensional preoperative imaging data, and the interoperative imaging system 130 is configured to provide a plurality of two-dimensional interoperative images from a plurality of different views.

### EXAMPLES

Further examples of some embodiments of the invention will now be described in detail. The broad concepts of the current invention are not intended to be limited to the particular examples.

### Example 1

FIG. 2 shows a fluoroscopy-guided robotic injection system 200 of some embodiments. The robotic injection system 200 is similar to the embodiments of the robotic injection system 100, 1100 discussed with respect to FIG. 1 and FIG. 11A, and like reference numerals have been used to refer to the same or similar components. A detailed description of these components will be omitted, and the following discussion focuses on the differences between these embodiments. Any of the various features discussed with any one of the embodiments discussed herein may also apply to and be used with any other embodiments.

The robotic injection system 200 can be used for transforaminal lumbar epidural injections. The robotic injection system 200 includes a robotic injection platform to perform planning, registration and navigation, automatic injection, and post-operative analysis. The envisioned workflow for robotically performed injections, shown in FIG. 2, is similar to the conventional clinical procedure. A clinician codifies his/her plan before the procedure by annotating target points and trajectories for needles on a CT image. The robotic injection system is registered to the patient's anatomy using multi-view X-ray 2D/3D registration [14]. The system 200 1) inserts the needle; 2) injects the steroid; 3) removes the needle; 4) verifies the injection; and 5) repeats the process as needed for each planned injection. The system setup, components and workflow are discussed in more detail below.

FIG. 2 shows the overall pipeline of the robotic injection system 200 in some embodiments. Inputs include (a) patient-specific CT scan and spine vertebrae segmentation, and (b) an injection device model. The planning module (c) shows the surgeon's interface to annotate needle injection trajectories and an example display of the planned trajectories on the CT segmentation. Multi-view registration presents: (d) multi-view C-arm X-ray projection geometries. The source-to-detector center projection line is rendered in green and the detector planes are rendered as squares. The needle injector guide and the spine anatomy are rendered using the registration pose. Panels (e), (f), and (g) show registration overlay images of the needle injector guide. The outlines of the reprojected injection device are overlayed in green. Panels (h), (i), and (j) show registration overlay images of the cadaveric spine vertebrae. An image of an actual cadaveric needle injection procedure is shown in panel (k).

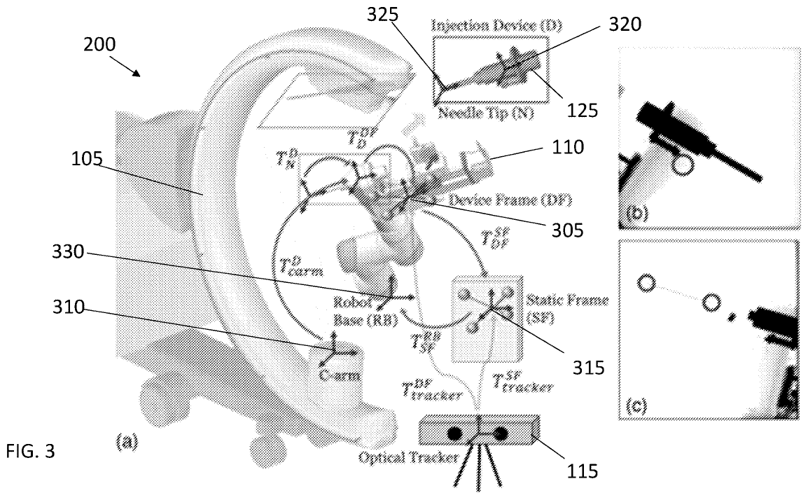

FIG. 3 shows a calibration scheme used in some embodiments for the robotic injection system 200. In panel (a) of FIG. 3, coordinate frames are marked as red cross arrows. These include a device frame 305 (denoted DF) for the spine injection robot 110, a C-arm frame 310 (denoted CARM) for the C-arm 105, a static frame 315 (denoted SF), an injection device frame 320 (denoted D) for the injection device 120, a needle tip frame 325 (denoted N) for the needle of the injection device 120, a robot base frame 330 (denoted RB) for the base of the robot, and a tracker frame 335 (denoted TRACKER) for the tracking system 115.

Key transformations between reference frames (each transformation denoted T, with subscript referring to source frame and superscript referring to target frame) are shown in blue arrows. Panel (b) shows an example X-ray image used for hand-eye calibration. Example BBs are marked in a red circle. Panel (c) shows an example X-ray image used for needle calibration. The needle tip and base points are marked in red circles. A 3D model of the injection device 120 for 2D/3D registration is illustrated in panel (d) on top.

An experiment performed using the robotic injection system 200 of some embodiments is now described. Needle targets and trajectories were planned in a custom-designed module in 3D Slicer [16]. Pre-procedure lower torso CT scans were acquired. The CT images were rendered in the module with the standard coronal, sagittal, and transverse slice views as well as a 3D volume of the bony anatomy, segmented automatically by Slicer's built-in volume renderer. Needle target and entry points could be picked on any of the four views. A model needle was rendered in the 3D view according to the trajectory defined by the mentioned points and the needle projection was displayed on each slice view. Users had the option to switch to a “down-trajectory” view where the coronal view was replaced with a view perpendicular to the needle trajectory and the other two slice views were reformatted to provide views orthogonal to the down-trajectory view. These views, together with 3D rendering, provided opportunities to determine the amount of clearance between the planned needle trajectory and bone outline. This module is provided in github (see https://github.com/htp2/InjectionTrajectoryPlanner). An example screenshot of the surgeon's interface is presented panel (c) of FIG. 2.

For the experiment, the robotic system's end effector consisted of a custom-designed automated injection unit [14], attached to a 6-DOF UR-10 (Universal Robots, Odense, Denmark). The forward kinematic accuracy of the spine injection robot 110 is insufficient for this task. This insufficiency is further amplified by the weight of the injection device 120 and long operating reach needed to perform injections on both sides of the subject's spine 125 from L2 to the sacrum from a single position at the bedside. To ameliorate these inaccuracies, an NDI Polaris (Northern Digital Inc., Waterloo, Ontario, Canada) system was used to achieve closed-loop position control of the robotic injection system 200. A custom-designed attachment between the syringe and needle was constructed to allow for the robotic injection system 200 to leave a needle behind after placement with minimal perturbation and to allow for repeatable reloading of needles with minimal positional deviation. (FIG. 1). The syringe mount consisted of a plug with a female Luer lock and a receptacle with a male Luer lock, for which the receptacle was screwed onto the syringe and the needle was screwed into the plug. The mating tapers on each Luer lock connection ensured concentricity between needles, while the linear degree of freedom between the plug and receptacle, when unlocked, allowed for precise adjustment of the needles' axial stick-out, to ensure that the length from the tip of the needle to the base of the injection device 120 was consistent between trials.

The robotic injection system 200 was navigated using pose estimations from X-ray image-based 2D/3D registration. An accurate calibration of the device registration model to the robot kinematic chain is required for automatic positioning of the spine injection robot 110 and injection. To achieve closed-loop navigation, several calibrations were required: hand-eye calibration of the optical frame, hand-eye calibration of the injection device and needle calibration (FIG. 3).

Hand-eye Calibration of the Device Frame: A hand-eye calibration was performed to determine the location of the optical tracker body on the injector unit coordinate frame (DI) relative to the robot's base coordinate frame (RB). This allowed for real-time estimation of the manipulator Jacobian Jₘ associated with movement of the injection device 120 attached to the base robot. The calibration was performed by moving the robot to 60 configurations within the region of its workspace in which the injections would occur, recording the robot's forward kinematics and the optical tracker body location (T_{SF}^{SF}), and using these measurements to solve an AX=XB problem to find T_{SF}^{RB}, as described in panel (a) of FIG. 3.

Hand-eye Calibration of the Injection Device: Another hand-eye calibration was conducted to compute the trans-formation of the injection device model coordinate frame (D) to the optical tracker unit. This transformation integrates the registration pose estimation to the closed-loop control. Metallic BBs were glued to the surface of the injection device 120 and their 3D positions were extracted in the model. At different robot configurations, X-ray images of the injection device 120 were acquired. 2D BB locations are easily detected on the images and were manually annotated, as described in panel (b) of FIG. 3. The rigid pose of the injection device (T_{Carm}^{D}) was estimated by solving a PnP problem. This also results in an AX XB problem to find T_{D}^{DF} in panel (a) of FIG. 3. In some embodiments, these two hand-eye calibration processes occur when the injector is removed and reattached to the spine injection robot 110.

Needle Calibration: As the positional accuracy of the needle tip is of greatest importance, a one-time calibration was also completed to determine the location and direction of the needle tip relative to the marker body on the injector. Ten X-ray images were taken with the injector and the needle in the view of the image. The needle tip and BB markers attached to the surface of the injector were annotated in each image, as described in panel (c) of FIG. 3. These annotations were used when solving the optimization of the 3D location of the needle tip relative to the injector's coordinate frame, which is described further below.

To this end, the chain of transformation connects the frame of the C-arm 105, the model of the injection device 120, the optical marker units, and the base frame of the spine injection robot 110. These calibration results are used to navigate the injector to the planning trajectories once the registration is complete.

### Intra-Operative Registration and Navigation

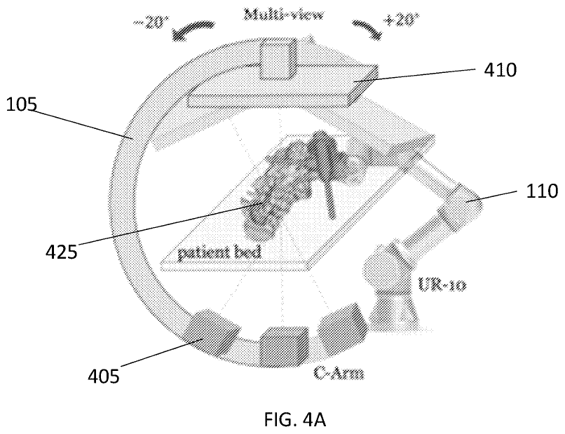

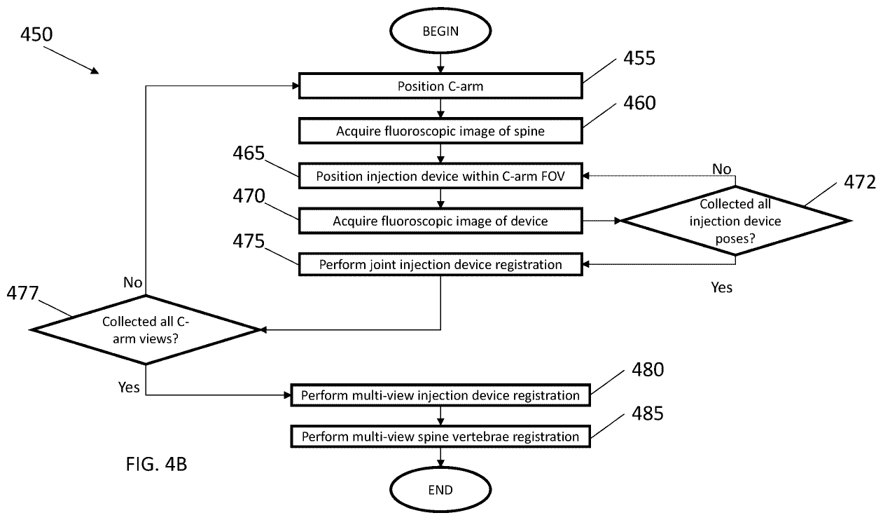

FIG. 4A shows an illustration of collecting multi-view C-arm images for the robotic injection system 200 of some embodiments. The source 405 and detector 410 of the C-arm 105 are shown in three positions separated by 20 degrees. The spine anatomy 425 is rendered on the patient bed. Various configurations of the injection device 120 and spine injection robot 110 are presented.

In some embodiments, the C-arm 105 was positioned at multiple geometric views with separate angles (for example, increments of ±20°). At each C-arm view, a fluoroscopic image was taken of the spine. Then, the injection device 120 was positioned at varied configurations above the patient anatomy and within the capture range (e.g., field of view) of the C-arm 105. The patient remained stationary during the registration phase, and the robot base was fixed relative to the patient bed. Fluoroscopic images of the injection device 120 were taken for each pose of the injection device 120. These robot configurations were saved and kept the same while the C-arm 105 was positioned at different views. A general data acquisition workflow is illustrated in FIG. 4B, which is described below.

FIG. 4B shows a process 450 for a multi-view registration workflow performed by the robotic injection system 200 of some embodiments. The process 450 details data acquisition and registration steps that are described in more detail below. The process 450 allows for intra-operative pose estimation of the injection device and the spine vertebrae, using multi-view X-ray image-based 2D/3D registration.

The process 450 begins at 455 by positioning the C-arm 105, and at 460, acquiring a fluoroscopic image of the spine anatomy 425.

At 465, the process 450 positions the injection device 120 within the field of view of the positioned C-arm 105.

At 470, the process 450 acquires a fluoroscopic image of the injection device 120. The process 450 determines, at 472, whether all injection device poses have been collected. If all injection device poses have not been collected, the process 450 returns to 465, to re-position the injection device 120 within the field of view of the positioned C-arm 105.

If all injection device poses have been collected, the process 450 proceeds to 475 to perform joint injection device registration.

At 477, the process 450 determines if all C-arm views have been collected. If all C-arm views have not been collected, the process 450 returns to 455 to re-position the C-arm 105 for another view.

If all C-arm views have been collected, the process 450 proceeds to 480 to perform multi-view injection device registration, and to 485 to perform multi-view spine vertebrae registration. These operations are described in more detail below.

The acquired fluoroscopic images are used in some embodiments for multi-view injection device registration and multi-view spine vertebrae registration, which are described in the following sections.

### Multi-View Injection Device Registration

Image intensity-based 2D/3D registration was performed to estimate the C-arm multi-view geometries and the intra-operative pose of the injection device (T_{Carm}^{ID}). Intensity-based 2D/3D registration optimizes a similarity metric between a target image and a digitally reconstructed radiograph (DRR) image simulated from the 3D injection device model (V^{D}) [14]. Because single-view 2D/3D registration is known to have severe ambiguity [15], a joint injection device registration was performed of various robot configurations at each C-arm view. Given J tracker observations T_{DF}_{₁}^{SF}, T_{DF}_{₂}^{SF}, . . . , T_{DF}_{_{J}}^{SF}, the hand-eye calibration matrix T_{ID}^{DF}, the injection device poses in the static frame are T_{ID}_{ⱼ}^{SF}=T_{ID}^{DF}·T_{DF}_{ⱼ}^{SF}, j∈{1, . . . J}. The first pose was used as reference and the rest of the poses can be computed relative to the reference pose using T_{ID}_{ₒ}^{ID}^{ⱼ}=T_{ID}^{DF}T_{DF}_{ⱼ}^{SF}·(T_{DF}_{₀}^{SF})⁻¹·(T_{ID}^{DF})⁻¹. Given an X-ray image Iₖ^{D}^{ⱼ} (the k^{th} C-arm view and the j^{th} injection device pose), a DRR operator (P), a similarity metric(S), the joint 2D/3D registration estimates the injection device pose (T_{Carm}_{ₖ}^{I D}^{₀}) by solving the following organization problem:

$${min}_{{T}_{{Carm}_{k}}^{I {D}_{0}} ∈ SE ( 3 )} {\sum }_{j = 1}^{J} S ( {I}_{k}^{{D}_{j}} , 𝒫 ( {V}^{D} ; {T}_{{Carm}_{k}}^{I {D}_{0}} , {T}_{ID}^{DF} , {T}_{{DF}_{j}}^{SF} ) ) . \qquad ( 1 )$$

The similarity metric (S) was chosen to be patch-based normalized gradient cross correlation (Grad-NCC) [17]. The 2D X-ray image was downsampled 4 times in each dimension. The optimization strategy was selected as “Covariance Matrix Adaptation: Evolutionary Search” (CMA-ES) due to its robustness to local minima [18]. The registration gives an accurate injection device pose estimation at each C-arm view (T_{Carm}_{ₖ}^{I D}^{₀}).

Because the injection device pose was the same when the C-arm view was changed, the pose functioned as a fiducial to estimate the multi-view C-arm geometry. Given the first C-arm view as reference, poses of the rest C-arm views can be calculated using T_{Carm}_{₀}^{Carm}^{ₖ}=(T_{Carm}_{ₖ}^{I D}^{₀})⁻¹·T_{Carm}_{₀}^{I D}^{₀}, k∈{1, . . . , K}, where K is the total number of C-arm views. A multi-view injection device registration was performed to estimate the reference injection device pose in the reference C-arm view (T_{Carm}_{₀}^{I D}^{₀}) by solving the optimization:

$${min}_{{T}_{{Carm}_{0}}^{I {D}_{0}} ∈ SE ( 3 )} {\sum }_{k = 1}^{K} S ( {I}_{k}^{{D}_{0}} , 𝒫 ( {V}^{D} ; {T}_{{Carm}_{0}}^{I {D}_{0}} , {T}_{{Carm}_{0}}^{{Carm}_{k}} ) ) . \qquad ( 2 )$$

The same similarity metric, image processing and optimization strategy was used as introduced in the joint injection device registration. T_{Carm}_{₀}^{I D}^{₀} was derived from multi-view registration further refined the result of joint registration under single-view. The multi-view C-arm geometries (T_{Carm}_{₀}^{Carm}^{ₖ}) were also used for multi-view spine vertebrae registration.

### Multi-View Spine Vertebrae Registration

The spine vertebrae (Vₘ^{S}, m∈{1 . . . M}, where M is the total number of vertebrae for registration, were segmented from the pre-operative CT scans using an automated method [19]. Intraoperative pose estimation of the spine vertebrae (T_{Carm}_{₀}^{v}^{ₘ}) was achieved following a coarse-to-fine manner. Firstly, a single-view rigid 2D/3D registration was performed using the first C-arm view X-ray image and the rigid vertebrae segmentation from the pre-operative CT scans. Given the X-ray image I₀^{S} (the first C-arm view X-ray image for spine registration), a DRR operator (P), a similarity metric(S), the single-view 2D/3D registration estimated the pose of rigid spine vertebrae (T_{Carm}_{₀}^{v}) by solving the following optimization problem:

$${min}_{{T}_{{Carm}_{0}}^{v} ∈ SE ( 3 )} {\sum }_{k = 1}^{K} S ( {I}_{0}^{S} , 𝒫 ( {\sum }_{m = 1}^{M} {V}_{m}^{S} ; {T}_{{Carm}_{0}}^{v} ) ) . \qquad ( 3 )$$

Because of the intra-operative spine vertebrae shape difference from the pre-operative CT scans and the ambiguity of single-view 2D/3D registration, T_{Carm}_{₀}^{v} solved from equation 3 is prone to be less accurate. Precise intra-operative vertebrae pose estimation was achieved by performing multi-view vertebra-by-vertebra 2D/3D registration. The pose of each individual vertebra was optimized independently. The multiple C-arm geometries (T_{Carm}_{₀}^{Carm}^{ₖ}) were estimated from the injection device registration. The registration was initialized by T_{Carm}^{v} and estimates deformable spine vertebrae poses (T_{Carm}_{₀}^{v}^{ₘ}, m∈{1 . . . . M}) by solving the optimization:

$${min}_{{T}_{{Carm}_{0}}^{{v}_{m}} ∈ SE ( 3 )} {\sum }_{k = 1}^{K} S ( {I}_{k}^{S} , 𝒫 ( {\sum }_{m = 1}^{M} {V}_{m} ; {T}_{{Carm}_{0}}^{{v}_{m}} , {T}_{{Carm}_{0}}^{{Carm}_{k}} ) ) . \qquad ( 4 )$$

The registration setup and optimization strategies in both single-view and multi-view spine registrations were the same as intensity-based injection device registration. Multi-view spine vertebrae registration functioned as an accurate local search of each vertebra component of the deformable spine object. The vertebrae pose estimation (T_{Carm}_{₀}^{v}^{ₘ}) and the injection pose estimation (T_{Carm}_{₀}^{I D}^{₀}) were both in the reference C-arm frame. Their relative pose can be computed, using T_{I D}_{₀}^{v}^{ₘ}=(T_{Carm}_{₀}^{I D}^{₀})⁻¹·T_{Carm}_{₀}^{v}^{ₘ}, m∈{1 . . . . M} was used to update the injection plan of each nearby vertebra and navigate the spine injection robot 110 to the injection position.

The target trajectory consisting of an entry point and a target point for the needle injection was transformed into the optical marker coordinate frame on the injector (DF) using the system calibration matrices. The spine injection robot 110 was controlled to a start position, which was a straight-line extension of the target needle path above the skin entry. Next, the needle injector was moved along the straight line to reach the target point. To ensure smooth motion, joint velocities θ^{·} were commanded to the spine injection robot 110. These velocities were chosen by θ^{·}=Jₘ⁻¹·v, where v represents the instantaneous linear and angular velocities that would produce a straight-line Cartesian path from the start to goal positions. This is the desired method of movement for needle insertion. The pose of the injector relative to a base marker was measured using the optical tracker. Once the needle reached the target point, the needle head was manually detached from the syringe mount. Then the spine injection robot 110 was moved back to the start position to finish this injection.

### Post-Op Evaluation

Post-operative CT scans were taken and the needle tip and base positions from the CT images were manually annotated. The metrics of target point error, needle orientation error, and needle tip position relative the safety zone of this application were reported. Considering the spine shape mismatch of the post-operative and pre-operative CT scans, a 3D/3D registration was performed of each vertebra from post-op to pre-op CT. The annotated needle point positions were transformed to the pre-operative CT frame for comparison.

The needle injection safety zone was defined combining the conventional safe triangle, located under the inferior aspect of the pedicle [20], and the Kambin's triangle [21], defined as a right triangle region over the dorsolateral disc. The annotation of the safety zone was performed on pre-operative CT scans under the instruction of experienced surgeons. The safety zone for each injection trajectory target was manually segmented in 3D Slicer. The needle tip positions were checked relative to these safety zones in the post-operative CT scans as part of the evaluation.

### Experiments and Results

In the following section, the results of system calibration and verification using simulation and cadaveric experiments for some embodiments are reported. Results are presented below, of testing the robotic injection system 200 of some embodiments with a series of simulations and cadaveric studies and comparing the robot performance with an expert clinician's manual injection.

For system calibration, needle and hand-eye calibration were performed. For navigation system verification, simulation studies and cadaveric experiments were performed. Lower torso CT scan images of a male cadaveric specimen were acquired for fluoroscopic simulation and spine vertebrae registration. The CT voxel spacing was resampled to 1.0 mm isotropic. Vertebrae S1, L2, L3, L4 and L5 were segmented. The X-ray simulation environment was set up to approximate a Siemens CIOS Fusion C-Arm, which has image dimensions of 1536×1536, isotropic pixel spacing of 0.194 mm/pixel, a source-to-detector distance of 1020 mm, and a principal point at the center of the image. X-ray images were simulated in this environment with known ground-truth poses (e.g., using the xreg library, https://github.com/rg2/xreg) and tested the multi-view registration pipeline.

### System Calibration

The needle base and tip positions were pre-operatively calibrated in the injection device model frame, using an example needle attached to the syringe mount. Six X-ray images were taken with variant C-arm poses. 2D needle tip, base (xₖ^{top}, xₖ^{base}, k∈{1, . . . , 6}) and metallic BB positions were manually annotated in each X-ray image (See panel (c) of FIG. 3). The C-arm pose ((T_{Carm}^{D})ₖ^{pnp}, k∈{1, . . . , 6}) was estimated by solving the PnP problem using corresponding 2D and 3D BBs on the injection device. Using the projector operator (P), the 3D needle tip and base positions (p_{D}^{tip}, p_{D}^{base}) were estimated by solving the following optimization:

$${min}_{{P}_{D}^{tip , base} ∈ {ℝ}^{3}} {\sum }_{k = 1}^{K} ( {x}_{k}^{tip , base} , 𝒫 ( {p}_{D}^{tip , base} , {( {T}_{Carm}^{D} )}_{k}^{pnp} ) ) . \qquad ( 5 )$$

The optimization was performed using brute force local search starting from a manual initialization point. The residual 2D error was reported by calculating the l₂ difference of the annotated needle tip and base points (x^{tip,base}) and the reprojected points (P(p_{D}^{tip,base}, (T_{Carm}^{D})ₖ^{pnp})) on each X-ray image. The mean 2D needle tip and base point errors were 0.64±0.53 mm and 0.57±0.42 mm, respectively.

The injection device 120 was moved to 30 variant configurations for injection device hand-eye calibration, while the C-arm 105 was fixed static. At each configuration, an X-ray image was taken and the injection device pose T_{Carm}^{D} was solved. After solving the AX=XB problem to find T_{D}^{DF}, the calibration accuracy was reported by calculating the injection device tip position difference between the PnP estimation ((T_{Carm}^{D})ᵢ^{pnp}) and estimation using the chain of calibration transformations:

$${( {T}_{Carm}^{D} )}_{i}^{cali} = {( {T}_{D}^{DF} )}^{- 1} \cdot {( {T}_{DF}^{SF} )}_{i}^{- 1} \cdot {T}_{DF}^{SF} \cdot {T}_{D}^{DF} \cdot {( {T}_{Carm}^{D} )}_{0} , \qquad ( 6 )$$

where i is the index of the calibration frame and (T_{Carm}^{D})₀ is our reference pose corresponding to the first calibration frame. The hand-eye calibration error was calculated as the mean l₂ difference of the estimated needle tip point in the injector model (p_{D}^{tip}) between these two pose estimations: ∥((T_{Carm}^{D})ᵢ^{cali}−(T_{Carm}^{D})ᵢ^{pnp})·p_{D}^{tip}∥. The mean error was 2.49±1.55 mm.

### Simulation Study

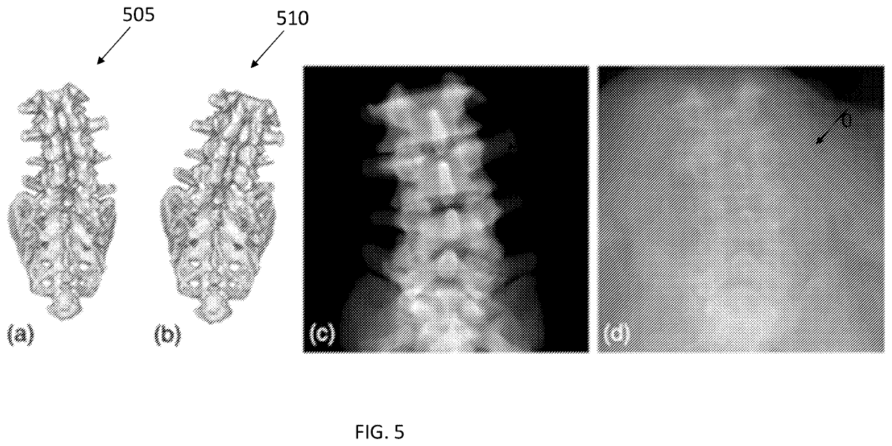

The registration performance was tested under various settings, including single-view and multi-view C-arm geometries, rigid spine and deformable spine, etc. One thousand simulation studies were performed with randomized poses of the injection device and the spine for each registration workflow. To simulate the intra-operative spine shape difference from the pre-operative CT scans, random rotation change was applied to the consecutive vertebrae CT segmentations. FIG. 5 presents an example of a simulated spine deformation in some embodiments. This deformed spine model was used to perform rigid spine registration and initialize the vertebrae pose in deformable spine registration. The reference frame of the injection device model was defined at the center of the injector tube. Reference frames of spine vertebrae were manually annotated at the center of each individual vertebra in the pre-operative CT scan. Thus, the registration poses, such as T_{Carm}^{ID}, T_{Carm}^{v} refer to the rigid transformations from the simulated C-arm source to these reference frames. The registration accuracy is reported based on simulated “ground truth” poses of the objects using

$$δ {T}_{Carm}^{obj} = {( {T}_{Carm}^{obj} )}_{gt} \cdot ( {T}_{Carm}^{obj} ) \frac{- 1}{regi} ,$$

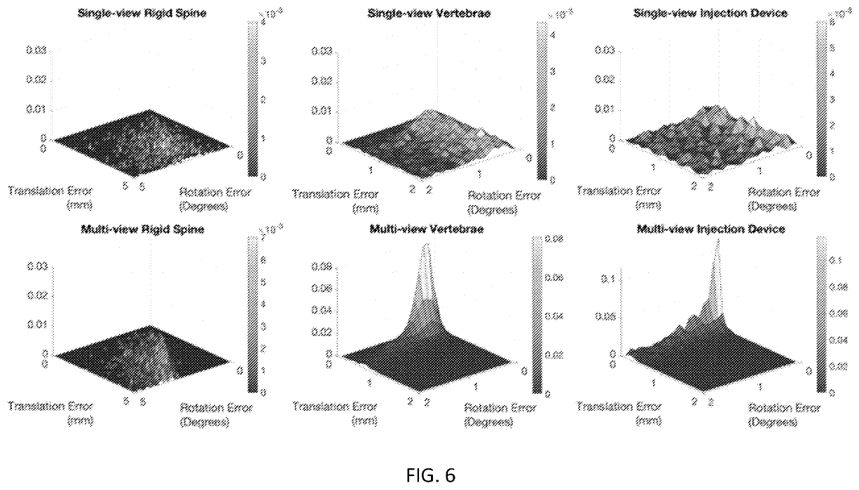

obj∈{ID, v}, where gt and regi refer to ground truth and registration estimation, respectively. The detailed simulation setup is described in the following subsections. Numeric results and statistical plots are presented in Table 1 and FIG. 6.

FIG. 5 presents an example of a simulated spine deformation in some embodiments. Panel (a) shows a rendered vertebrae segmentation 505 from pre-operative CT scans. Panel (b) shows an example of randomly simulated spine shape 510. Panel (c) shows an example DRR image 515 of the spine vertebrae. Panel (d) shows an example simulation X-ray image 520.

TABLE 1
Mean Registration Error in Simulation Study

| | | Translation Error | Rotation Error |
|---|---|---|---|
| | | (mm) | (degrees) |
| Single-View | Rigid Spine | 4.79 ± 2.36 | 2.79 ± 1.70 |
| | Vertebrae Injection | 3.50 ± 2.91 | 1.05 ± 1.88 |
| | Device | 2.15 ± 1.57 | 1.62 ± 1.40 |
| Multi-View | Rigid Spine | 3.69 ± 1.60 | 2.89 ± 1.23 |
| | Vertebrae Injection | 0.76 ± 0.28 | 0.88 ± 0.68 |
| | Device | 0.17 ± 0.60 | 1.21 ± 1.31 |

### Single-View Registration

2D/3D registration workflows were performed of rigid spine, deformable spine and injection device by simulating single-view X-ray images. For every registration running, uniformly sampled rotations from −5 to 5 degrees in all three axes were applied to the vertebrae segmentations. Random initializations of the spine and injection device were uniformly sampled including translation from 0 to 10 mm and rotation from −10 to 10 degrees. Table I summarizes the magnitudes of translation and rotation errors. The vertebrae error is computed as a mean error of vertebra S1, L2, L3, L4 and L5. A mean translation error was achieved of 3.50±2.91 mm and a mean rotation error of 1.05±1.88 degrees using vertebra-by-vertebra registration, and 2.15±1.57 mm, 1.62±1.40 degrees for injection device registration, respectively.

### Multi-View Registration

Three multiple C-arm pose geometries were estimated with a uniformly sampled random separation angle between 20 and 25 degrees for the two side views. The three registration workflows tested in single-view were performed with the same settings under this multi-view setup. Both the vertebrae and the injection device registration accuracy was improved. The mean vertebra registration error was 0.76±0.28 mm and 0.88±0.68 degrees in translation and rotation respectively, and the injection device registration error was 0.17±0.60 mm and 1.21±1.31 degrees, respectively. Joint histograms of the translation and rotation errors are presented in FIG. 6. From the plots, the multi-view vertebrae and injection device registration were clearly observed to have the best error distribution with the cluster close to zero errors.

FIG. 6 shows normalized 2D histograms of registration pose error (∂T_{Carm}_{₀}^{1D}), (∂T_{Carm}_{₀}^{v}) reported in joint magnitudes of translation and rotation, for the robotic injection system of some embodiments.

### Cadaver Study

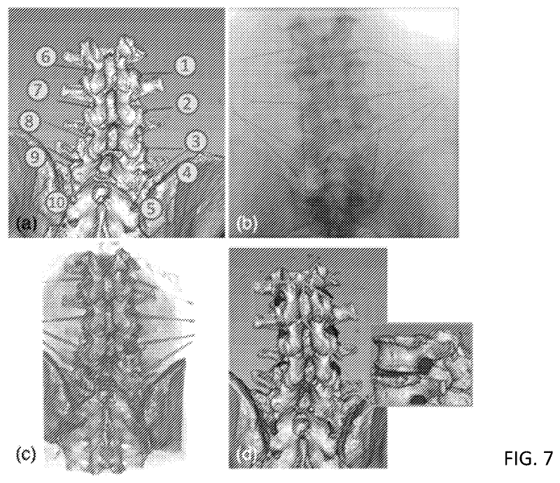

FIG. 7 shows an example of robotic injection using the robotic injection system 200 of some embodiments, on a cadaver specimen. Panel (a) shows a screenshot of planning trajectories. Panel (b) shows an example X-ray image taken after the robotic needle injections. Panel (c) shows a rendering of the post-operative CT scans. Panel (d) shows an illustration of a manual safety zone.

The injection plan on a cadaver specimen was made by an expert clinician who also performed the procedure according to the plan shown in panel (a) of FIG. 7, allowing for comparison of performance to the robotic injection. Ten injections were simulated via needle placement at five targets on each side of the specimen. Targets were the epidural spaces L2/3, L3/4, L4/5, L5/S1, and the first sacral foramen on each side. The target points were planned at the center of each safety zone.

Needle injection was performed with the robotic injection system 200 according to the injection plan under X-ray image-based navigation. The registration workflow was initialized using the PnP solutions from eight corresponding 2D and 3D anatomical landmarks. 3D landmarks were annotated pre-operatively on the CT scans. 2D landmarks were annotated intra-operatively after taking the registration X-rays. For the purpose of needle placement validation in this study, a small deviation from the proposed clinical workflow was performed in which needles were left within the specimen after placement. This allowed for acquisition of a post-procedure CT to directly evaluate the needle targeting performance relative to the cadaveric anatomy with high fidelity. After the post-operative CT scan was taken, needles were removed and the needle placement was repeated by the expert clinician as their normal operation, using fluoroscopy as needed and another post-procedure CT was taken for evaluation. FIG. 7 presents rendering of the post-operative CT scan in panel (c) and an X-ray image in panel (b) taken after the robotic injection.

The needle injection performance was reported using three metrics: needle tip error, needle orientation error, and safety zone. The needle tip error was calculated as the l₂ distance between the planned trajectory target point and the injected needle tip point after registering vertebrae from post-operative CT to pre-operative CT. The orientation error was measured as the angle between trajectory vectors pointing along the long axis of the needle in its measured and planned positions. The results are summarized in Table 2. The robotic needle injection achieved a mean needle tip error of 5.09±2.36 mm and mean orientation error of 3.61±1.93 degrees, compared to the clinical expert's performance of 7.58±2.80 mm and 9.90±4.73 mm, respectively. The manually annotated safety zones in the post-operative CT scans are illustrated in panel (d) of FIG. 7. All the injected needle tips, including both the robotic and clinician's injections, were within the safety zones.

TABLE 2
Cadaveric Needle Injection Accuracy

| | Needle Tip Error (mm) | | Orientation Error (degrees) | |
|---|---|---|---|---|
| ID | Robot | Surgeon | Robot | Surgeon |
| 1 | 3.13 | 9.46 | 5.13 | 5.77 |
| 2 | 6.13 | 11.35 | 1.85 | 8.30 |
| 3 | 7.02 | 6.17 | 2.40 | 13.20 |
| 4 | 7.14 | 12.29 | 4.60 | 6.98 |
| 5 | 4.36 | 6.88 | 2.06 | 9.28 |
| 6 | 1.54 | 8.46 | 1.56 | 8.61 |
| 7 | 5.14 | 3.36 | 2.63 | 7.05 |
| 8 | 8.01 | 7.02 | 5.52 | 20.88 |
| 9 | 1.57 | 5.28 | 2.95 | 13.46 |
| 10 | 6.85 | 5.56 | 7.37 | 5.51 |
| Mean | 5.09 ± 2.36 | 7.58 ± 2.80 | 3.61 ± 1.93 | 9.90 ± 4.73 |

### Discussion

In some embodiments, the robotic injection system 200 is fiducial-free, using purely image information to close the registration loop, automatically position the needle injector to the planned trajectory, and execute the injection. The robotic needle injection was navigated using multi-view X-ray 2D/3D registration. For this application, the simulation study has shown that multi-view registration is significantly more accurate and stable than single-view registration in all the ablation registration workflows (Table. I). This is because multi-view projection geometries fundamentally improve the natural ambiguity of single-view registration. The specially designed vertebra-by-vertebra registration solves the problem of spine shape deformation between pre-operative CT scan and intra-operative patient pose. In simulation, the mean multi-view registration error decreased from 3.69±1.60 mm, 2.89±1.23 degrees to 0.76±0.28 mm, 0.88±0.68 degrees in translation and rotation, using pre-operative rigid spine segmentation compared to multiple vertebrae.

The cadaver study experiments show the feasibility of using the system for transforaminal lumber epidural injections. The comparison study with an expert clinician's manual injection using the same plan presents clear improvements in both translation and orientation accuracy: mean needle tip translation error of 5.09±2.36 mm and 7.58±2.80, mean needle orientation error of 3.61±1.93 degrees and 9.90±4.73 degrees, corresponding to the robot and clinician's performance, respectively. The performance was also evaluated using the defined safety zone for this application. Both the robotic and clinician's injected needle tips laid inside the safety zones. Although the expert clinician's injection tip error and orientation error are larger, this manual injection's accuracy is still sufficient for this application. However, the robotic performance of higher accuracy and stability demonstrates potential reduction of the risk of violating the safety zone.

The individual contributions of errors due to hand-eye calibration and registration were also considered. The needle tip error due to registration as compared to planning was 2.82±2.61 mm. The needle tip error resulting from hand-eye calibration was 2.49±1.55 mm. Two other factors affecting the overall error are 1) the needle tip deflected slightly due to the relatively large distance between the tip and the end effector; and 2) calibration was performed only for one needle and did not repeat for successive injections with different needles. In the future, the size of the injection unit can be optimized to reduce the mentioned effect. Calibrations after changing each needle may also help to reduce the reported translation error.

One common concern of the fluoroscopic navigation system is the excessive radiation exposure to the patient. The approach of some embodiments requires ten to twelve X-rays to register the patient to the injection device 120. Considering X-rays are commonly used in the clinician's manual injections to check the needle position, this amount of radiation is acceptable for this procedure. The pipeline is designed to be fully automated, however, the current implementation required a few manual annotations from the clinician to initialize the registration. Future work would consider automating the intra-operative landmark detection to further simplify the workflow, similar to work reported in [22], [23].

In this study, needle steering was neglected. This is a widely studied topic, and such functionality could be added in future work and may improve results. The decision to not consider needle steering was made as 1) the focus of this work was on the novel application of the registration techniques used to the spine and correction via needle steering could mask inaccuracies of the registration; 2) The relatively large injection target area does not necessitate sub-millimeter accuracy; and 3) the use of stiff low gauge needles in this application limits bending in soft tissue, reducing both the need for, and the effect of needle steering.

### Conclusion

In this example, a fluoroscopy-guided robotic injection system of some embodiments is presented. The workflows of using multi-view X-ray image 2D/3D registration are shown to estimate the intra-operative pose of the injection device and the spine vertebrae. System calibration was performed to integrate the registration estimations to the planning trajectories. The registration results were used to navigate the robotic injector to perform automatic needle injections. The system was tested with both simulation and cadaveric studies, and involved comparison to an experienced clinician's manual injections. The results demonstrated the high accuracy and stability of the proposed image-guided robotic needle injection.

### Example 2

The autonomous spinal robotic injection system 200 of some embodiments was used for a proof-of-concept study of transforaminal lumbar epidural injections. The aim of the study was to demonstrate a proof-of-concept model for the use of an autonomous robotic controlled injection delivery system as it pertains to safety and accuracy of lumbar transforaminal epidural injections. The purpose of this study was to compare the accuracy of freehand transforaminal epidural injections by an expert provider to the spinal robotic injection system 200 on a phantom model. The hypothesis was that the robotic injection system 200 would have a higher degree of accuracy compared to the conventional freehand method by the expert provider.

### Materials and Methods

### Study Design Overview

In this phantom study, 20 transforaminal epidural injections were performed; 10 using a freehand transforaminal procedure under fluoroscopic guidance by 1 expert provider and 10 using the robotic injection system 200. To determine sample size, an a priori power analysis was performed using the statistical power analysis program G*power 3.1, including a t-test, an alpha set at 0.05, an effect size Cohen's d=1.4, and a power of 0.8.¹¹ This resulted in a total sample size of 20, or 10 injections per group. A custom software pre-operative planning module was developed for this study where the provider was able to plan their ideal transforaminal needle trajectories in a 3D space. These pre-operative trajectories were then compared to the actual physical trajectories performed by the provider and the robotic system. The primary metrics of the study were the distance and angulation between the pre-operative planned and actual post-operative needle tips and trajectories.

### Phantom

The phantom of the lumbosacral spine was made using a radiopaque adult-size spine model consisting of only bony elements from T12-sacrum (Sawbones, Washington, USA).¹² Sugar-free Metamucil (P&G, Cincinnati, OH, USA) was also added to ensure that the gelatin layer was opaque.¹³ Thus, the bone, needles, and targets were only visible with fluoroscopy and CT images, and not with the naked eye. A CT image of this phantom model was then acquired.

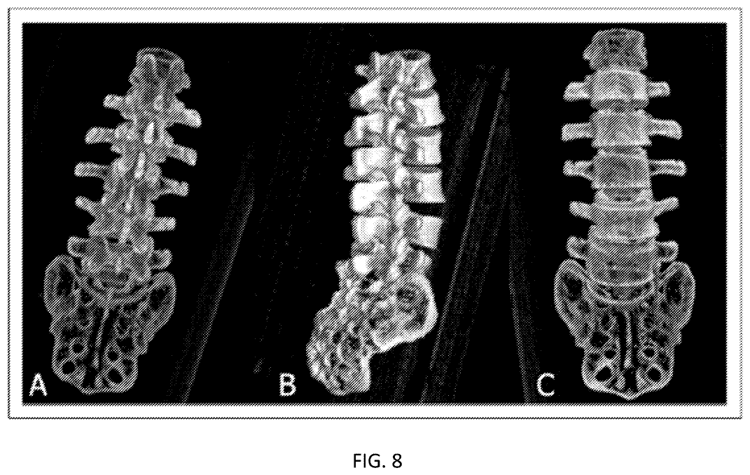

FIG. 8 shows a CT reconstruction of a radiopaque sawbones lumbar spine model used in some embodiments. Panel (a) shows a Posterior view 805 of the model, panel (b) shows an oblique view 810 of the model, and panel (c) shows an anterior view 815 of the model.

### Pre-Operative Planning Software

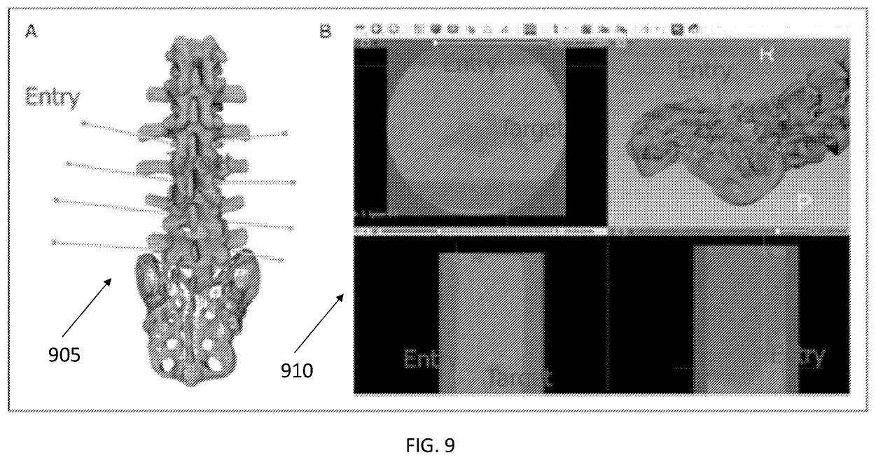

A custom pre-operative planning module was developed for this study using 3D Slicer.¹⁴ In this software module, needle trajectories could be planned on a CT image (FIG. 9 described below). The CT volume was displayed with standard anatomical slices as well as with a 3D rendering. A plan was created by placing a target point at the intended needle tip position during injection, and an entry point where the needle should enter the body. This plan was created by an expert interventional pain management provider for all trajectories. This was made for bilateral foraminal trajectories targeting L2/3, L3/4, L4/5, L5/S1 and S1 foramina, for a total of 10 “ideal” trajectories. With a transforaminal posterolateral approach, the final needle tip position on the axial view in this pre-operative planning module was located within the posterior margin of the neural foramen.⁴ A model of the needle, and a line representing its trajectory, was displayed on slice and 3D views, and an option to view the volume resliced down the axis of the injection was provided to visualize and confirm a collision-free trajectory. These representations were updated dynamically with any change in the planned points.

FIG. 9 shows an example of preoperative software modeling in some embodiments. Panel (a) shows a segmented CT image 905 of the phantom lumbar spine model demonstrating pre-operative planning for transforaminal epidural injections for bilateral L2/3, L3/4, L4/5, L5/S1 and S1 trajectories. Panel (b) demonstrates the custom software application 910 for selecting entry and target points on the phantom model CT image.

### Provider Technique

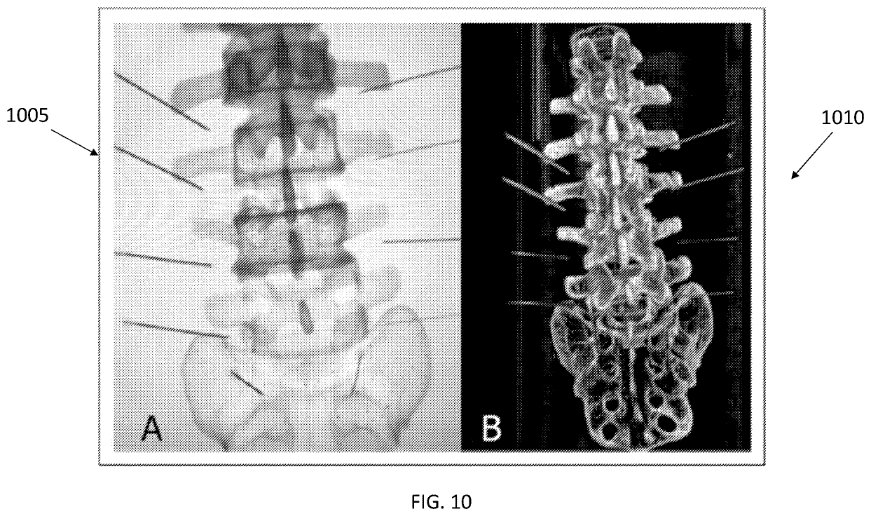

The expert interventional pain management provider performs transforaminal epidural spinal injections on over 500 patients per year and prefers performing injections under fluoroscopic guidance. A lateral oblique was obtained first to confirm needle entry site, slightly more inferior than the traditional safe triangle approach.⁴ A lateral radiograph was then taken to confirm the needle tip position with the needle tip remaining in the posterior half of the neural foramen, followed by an anteroposterior view (FIG. 10, panel A). This was performed for bilateral L2/3, L3/4, L4/5, L5/S1 and S1 trajectories, for a total of 10 injections. A post-operative CT image of this phantom model with the needles was then obtained (FIG. 10, panel B).

FIG. 10 shows freehand needle placement according to some embodiments. Panel (a) shows an anteroposterior radiograph 1005 of the phantom model after the freehand technique demonstrating needle tip and trajectory relationship to the radiopaque sawbones lumbar spine. Panel (b) shows a CT image 1010 of the phantom model with the needles in place.

### Robotic Technique

The needles were subsequently withdrawn from the phantom model and this process was repeated with the robotic targeting system. For the robotic technique, a UR10 robotic arm (Universal Robots, Odense, Denmark) was used with an attached custom-built injection device. The preoperative phantom spine CT images were acquired and were digitally segmented. Anatomical landmarks, such as the spinous and transverse process, were manually annotated in CT images. The injection device was pre-calibrated to the robot arm end effector. The spine phantom and the robotic injection device were kept static during registration. Intraoperative imaging of the spine and the injection device with radiographs were then obtained in multiple viewpoints, and corresponding anatomical landmark targets of the spine were then annotated. The corresponding anatomical landmarks are used to estimate an initial pose of the phantom model in the C-arm source frame by solving a PnP problem.¹⁵ A marker-less 2D/3D pipeline for registration was obtained.¹⁶ A 2D/3D image-based registration algorithm was then used to produce spine and injector pose estimations with respect to the extrinsic imaging device, the C-arm (see FIG. 11). The 3D phantom model and the robot injection device model are jointly registered by optimizing a similarity score between the simulated digitally reconstructed radiograph˜(DRR) images and the real X-ray images. The similarity metric was chosen to be patch-based normalized gradient cross correlation (Grad-NCC).¹⁷ The optimization strategy was selected as “Covariance Matrix Adaptation: Evolutionary Search” (CMA-ES) due to its robustness to local minima.¹⁸ The relative pose transformation between the phantom and the robot is obtained from the registration outcomes. This transformation is integrated to the robotic kinematics chain using the robot pre-calibration result. The robotic arm was then utilized to precisely orient the injection device. Using the location information from the registration, the robot was automatically moved to align with the planned trajectory as defined by the skin entry and target needle tip position in the planning software. The robot then inserted the needle along this trajectory, to the target point (FIG. 11).¹⁹ After all 10 needles were placed, an additional CT of the phantom model with the 10 spinal needles was obtained. The placement of the first needle did not influence placement of subsequent needles, as both the provider and robotic arm did not bump into or alter the trajectories of previously placed needles.

FIG. 11A shows a robotic injection system 1100 of some embodiments. The robotic injection system 1100 is similar to the embodiments of the robotic injection system 100, 200 discussed above with respect to FIG. 1 and FIG. 3, and like reference numerals have been used to refer to the same or similar components. A detailed description of these components will be omitted, and the following discussion focuses on the differences between these embodiments. Any of the various features discussed with any one of the embodiments discussed herein may also apply to and be used with any other embodiments.

FIG. 11A shows a schematic of the relationship for registration between the spine phantom model 1102, the C-arm 1105, and the robotic arm 1110 with the injection device 1120. FIG. 11B shows an image of the robotic arm 1110 with the attached injection device 1120.

### Statistical Analysis

The post-operative CT images from the freehand fluoroscopic guidance technique and the robotic technique were then incorporated into the 3D Slicer software and compared with the pre-operative trajectory plan. Procedural accuracy, defined as the absolute difference between pre-operative planning and actual post-operative needle tip distance (mm) in 3D space and angular orientation (degrees), were assessed between the freehand and robotic procedures utilizing independent Student's t test, with statistical significance set at P<0.05. For needle tip distance measurements, precision was reported by the range or difference between the lowest and highest absolute distances in 3D space within the freehand and robotic technique groups as compared to their ideal target points. Analyses were performed using SPSS, version 23.0, software (IBM Corp. Chicago, IL, USA).

TABLE 3
Comparison of Needle Tip Error Distance Between Freehand
Fluoroscopic Technique vs Robotic Technique

| | Freehand Fluoroscopic | | | | |
|---|---|---|---|---|---|
| Transformanimal | Technique | | Robotic Technique | | |
| Injection Level | Left | Right | Left | Right | P-Value |
| L2/L3 | 16.29 | 18.68 | 16.01 | 9.58 | |
| L3/L4 | 18.63 | 26.24 | 15.08 | 16.28 | |
| L4/L5 | 22.87 | 23.70 | 9.49 | 8.91 | |
| L5/S1 | 10.66 | 24.45 | 15.56 | 8.23 | |
| S1 | 15.09 | 23.91 | 8.43 | 6.14 | |
| Average error | 20.05 (4.99) | | 11.37 (3.88) | | <.001 |
| (SD) | | | | | |

### Results

Table 3 demonstrates the needle tip distance (mm) of the post-operative robotic and freehand technique compared to the pre-operative plan. All numeric values represent needle tip distance (mm) error between the actual needle and the planned trajectory. SD stands for standard deviation, and bold denotes statistical significance.

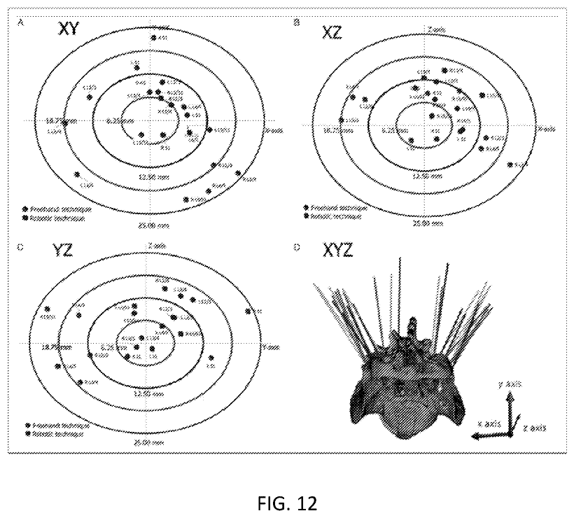

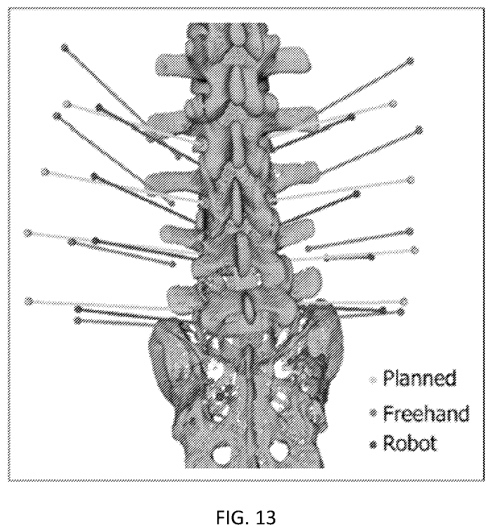

Procedural accuracy for robotically placed transforaminal epidural injections was significantly higher with the difference in pre- and post-operative needle tip distance being 20.1 (±5.0) mm in the freehand procedure and 11.4 (±3.9) mm in the robotically placed procedure (P<0.001, Table 3). Needle tip precision for the freehand technique was 15.6 mm (26.3-10.7) compared to 10.1 mm (16.3-6.1) for the robotic technique (FIG. 12). Differences in needle angular orientation deviation were 5.6 (±3.3) degrees in the robotically placed procedure and 12.0 (±4.8) degrees in the freehand procedure (P=0.003) (Table 4, FIG. 13).

Table 4 demonstrates the trajectory angulation error distance (degrees) of the post-operative robotic and freehand technique compared to the pre-operative plan. All numeric values represent trajectory angulation (degrees) error between the actual needle and the planned trajectory. SD stands for standard deviation, and bold denotes statistical significance.

FIG. 12 shows a scatter plot demonstrating differences in precision of the needle tip in mm between the postoperative freehand fluoroscopic (red) and robotic technique (blue) of some embodiments. Panel (a) shows the x- and y-axis (Axial view), panel (b) shows the x- and z-axis (AP View), and panel (c) shows the y- and z-axis (Sagittal view). Panel (d) denotes the orientation of the XYZ plane in relation to the phantom model. L and R denote Left and Right followed by the level of the transforaminal injection. Each circular ring is spaced out by 6.25 mm with a total diameter of 25 mm of the outer circle.

FIG. 13 shows differences from planned trajectories according to some embodiments. Differences in trajectories between the pre-operative planned software trajectories (yellow) and the actual post-operative freehand (red) and robotic techniques (purple) are demonstrated.

TABLE 4
Comparison of Trajectory Angulation Error Distance Between
Freehand Fluoroscopic Technique vs Robotic Technique

| | Freehand Fluoroscopic | | | | |
|---|---|---|---|---|---|
| Transformanimal | Technique | | Robotic Technique | | |
| Injection Level | Left | Right | Left | Right | P-Value |
| L2/L3 | 19.95 | 16.36 | 9.00 | 6.85 | |
| L3/L4 | 17.80 | 11.74 | 10.20 | 10.00 | |
| L4/L5 | 11.11 | 4.61 | 3.16 | 4.47 | |
| L5/S1 | 6.67 | 12.26 | 4.11 | 2.02 | |
| SI | 9.65 | 9.69 | 1.26 | 4.56 | |
| Average error | 11.98 (4.84) | | 5.56 (3.26) | | .003 |
| (SD) | | | | | |

### Discussion

Robotic-assisted surgical treatment continues to gain popularity for a variety of fields including general surgery, urology, orthopedics, and spine surgery. Here a proof-of-concept model is demonstrated for the use of an autonomous robotic controlled injection delivery system for enhancing the safety and accuracy of lumbar transforaminal epidural injections.

There is limited literature on the use of robotics for guiding spinal injections. In 2016, Beyer et al. performed a phantom model experiment for comparing robot-assisted to freehand facet joint puncture using the iSYS 1.3 (iSYS Medizintechnik GmbH, Kitzbuehel, Austria) robotic targeting system.²⁰ They demonstrated that robot-assisted puncture of the facet joints allowed more accurate positioning of the needle with a lower number of needle readjustments.²⁰ Additionally, Li et al. demonstrated the use of a body-mounted robotic system for Magnetic Resonance Imaging (MRI) guided lumbar spine injections within a closed bore magnet.²¹ They demonstrated, through a cadaveric study, that a robot-assisted approach is able to provide more accurate and reproducible cannula placements than the freehand procedure, as well as a reduction in the number of insertion attempts.²¹ Unlike the robotic injection system 1100 of some embodiments, their robotics system relied on radiopaque markers for registration, provided a semi-autonomous system by providing needle guidance to the correct location while the provider manually inserted the needle, and did not compare post-operative trajectories to pre-operatively planned trajectories. The robotic injection system 1100 demonstrates the autonomous entry of the needle to the desired depth and target point, rather than serving as a guide or tube holder for manual insertion.

There is currently no commercially available robotic platform system that can administer spine injections. However, robotics has started to gain popularity in the field of spine surgery by aiding in the placement of pedicle screws. The first commercial application was in 2004 with the SpineAssist (Mazor Robotics Ltd., Caesarea, Israel).²² Since then, other iterations of spinal robotic systems have been developed such as the Renaissance® (Mazor Robotics Ltd., Caesarea, Israel) in 2011 and Mazor X® (Mazor Robotics Ltd., Caesarea, Israel) in 2016. Additional commercial competitors include: ROSA® SPINE (Zimmer Biomet Robotics, Montpellier, France) in 2016 and the Excelsius GPS® (Globus Medical, Inc., Audubon, Pennsylvania) in 2017.²³ Skilled control of robot-assisted spine surgery has been shown to improve the accuracy of pedicle screw placement and decrease radiation exposure to surgical staff.²³ However, current robotic technology has many disadvantages including high cost, steep learning curves, semi-autonomous nature, limited surgical indications, and technological glitches.^{23,24}

Currently, robot-assisted spine surgery is mainly restricted to instrumentation procedures with pedicle screw insertion. All these systems are semi-active robotic systems. Meaning, they will guide and assist the surgeon in placing spinal implants, as opposed to a fully automatic system that performs the surgical operation autonomously.²² Hence, once aligned, the surgeon will then utilize a combination of guidewires, drills, and dilators to place pedicle screws manually to a desired depth.²³ The robotic injection system 1100 expands the robotic framework by making the entire process autonomous in nature so that the provider does not have to manually advance and inject the needle to a desired target. The robotic injection system 1100 not only guides the injection to the correct location but also controls for depth. Exact positioning of the needle with minimal 3D deviation from the pre-operatively planned trajectory might increase the therapeutic efficacy of epidural injections. For example, a provider may attempt to target the traditional safe or Kambin's triangle to administer an epidural injection. However, their needle tips may not reach this desired anatomical location.

In order to have an acceptable clinical outcome, the needle tip must be placed anywhere within a triangle shaped boundary as determined in the safe triangle, posterolateral, or Kambin's triangle approach.⁴ If the needle tip is within this triangular boundary, the injection should theoretically provide relief.⁴ For reference, Kambin's triangle height and width from L1-L5 ranges from 12-18 mm and 10-12 mm respectively, or an area of 60-108 mm² in the lumbar spine.²⁵ The present study has shown improved accuracy by the robotic platform which translates to appropriate needle placement directly resulting in improved patient outcomes. Further clinical studies must be conducted to confirm this benefit.

In addition to being autonomous, the proposed robotic injection system 1100 further advances spine robotics because it is also marker less or fiducial-less. In general, current spine robotics systems require a preoperative or intraoperative CT scan of the spine. In the operating room, a bone pin fiducial marker is placed on the patient. An intraoperative imaging device, such as the O-Arm® (Medtronic Sofamor Danek, Inc., Memphis, TN, USA), or other form of imaging, such as radiographs are utilized to capture both the surgical area of interest and the fiducial marker. This is used to perform a registration of the intraoperative imaging to the pre-operative CT scan and produce an intraoperative pose estimation. The surgeon can then plan a 3D trajectory on the reconstructed images and the robot will be able to align with this preplanned trajectory. However, if the fiducial markers are accidentally displaced during surgery, the robot would register this as movement by the patient and this would result in improper screw placement.

Some limitations of this study are that it utilized a phantom model, the small number of needle injections performed, and that a single expert provider was utilized for all freehand injections and trajectory planning. The distance that the experienced provider missed the pre-operative target by, as compared to the robotic system, could have been caused by the discrepancy in tactile feedback between the sawbones model compared to an actual patient that the provider is accustomed to performing the procedure on. However, fluoroscopy was ultimately used to determine the placement of the needle tip by the provider. Although all post-operative trajectories (freehand and robotics groups) were compared to the provider's ideal pre-operative trajectory planning on the software module, it is still highly dependent on the experience of the provider and might therefore vary considerably. Ideally, the robotic system would have been able to target each planned trajectory point flawlessly with no errors. Errors within the hand-eye calibration, the 2D-3D registration, and the hollow needle-steering and gelatin interface may have accounted for some errors. Additionally, the phantom model must be static while taking intraoperative radiographs for registration in our model. Future work will include refining registration and accounting for patient movement.

This study indicates that robotic assistance may be beneficial in enhancing the accuracy of transforaminal epidural injections. Although there are still many challenges, the potential of a marker less autonomous spinal robotic system of some embodiments has been demonstrated.

The terms “light” and “optical” are intended to have broad meanings that can include both visible regions of the electromagnetic spectrum as well as other regions, such as, but not limited to, infrared and ultraviolet light and optical imaging, for example, of such light.

The terms “computer”, “server”, “processor”, and “memory” all refer to electronic or other technological devices. These terms exclude people or groups of people. As used in this specification, the terms “computer readable medium,” “computer readable media,” and “machine readable medium,” etc. are entirely restricted to tangible, physical objects that store information in a form that is readable by a computer. These terms exclude any wireless signals, wired download signals, and any other ephemeral signals.

The term “computer” is intended to have a broad meaning that may be used in computing devices such as, e.g., but not limited to, standalone or client or server devices. The computer may be, e.g., (but not limited to) a personal computer (PC) system running an operating system such as, e.g., (but not limited to) MICROSOFT® WINDOWS® available from MICROSOFT® Corporation of Redmond, Wash., U.S.A. or an Apple computer executing MAC® OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but not limited to) a hard disk drive and/or a removable storage drive, representing a floppy diskette drive, a magnetic tape drive, an optical disk drive, a read-only compact disk (CD-ROM), digital versatile discs (DVDs), flash memory (e.g., SD cards, mini-SD cards, micro-SD cards, etc.), read-only and recordable Blu-Ray® discs, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In some embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

Some embodiments include electronic components, such as microprocessors, storage and memory that store computer program instructions in a machine-readable or computer-readable medium (alternatively referred to as computer-readable storage media, machine-readable media, or machine-readable storage media). The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

The computer may also include an input device may include any mechanism or combination of mechanisms that may permit information to be input into the computer system from, e.g., a user. The input device may include logic configured to receive information for the computer system from, e.g., a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or another camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions, including application-specific integrated circuits (ASICs) and field-programmable gate arrays (FPGAs). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The term “network” is intended to include any communication network, including a local area network (“LAN”), a wide area network (“WAN”), an Intranet, or a network of networks, such as the Internet.

The term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

In addition, at least one figure conceptually illustrates a process. The specific operations of this process may not be performed in the exact order shown and described. The specific operations may not be performed in one continuous series of operations, and different specific operations may be performed in different embodiments. Furthermore, the process could be implemented using several sub-processes, or as part of a larger macro process.

### REFERENCES (EXAMPLE 1)

[1] J. K. Freburger, G. M. Holmes, R. P. Agans, A. M. Jackman, J. D. Darter, A. S. Wallace, L. D. Castel, W. D. Kalsbeek, and T. S. Carey, “The rising prevalence of chronic low back pain,” Archives of internal medicine, vol. 169, no. 3, pp. 251-258, 2009.
[2] B. Duthey, “Background paper 6.24 low back pain,” Priority medicines for Europe and the world. Global Burden of Disease (2010), (March), pp. 1-29, 2013.
[3] V. B. Vad, A. L. Bhat, G. E. Lutz, and F. Cammisa, “Transforaminal epidural steroid injections in lumbosacral radiculopathy: a prospective randomized study,” Spine, vol. 27, no. 1, pp. 11-15, 2002.
[4] G. Li, N. A. Patel, W. Liu, D. Wu, K. Sharma, K. Cleary, J. Fritz, and I. Iordachita, “A fully actuated body-mounted robotic assistant for MRI-guided low back pain injection,” in 2020 IEEE International Conference on Robotics and Automation (ICRA). IEEE, 2020, pp. 5495-5501.
[5] G. Li, N. A. Patel, J. Hagemeister, J. Yan, D. Wu, K. Sharma, K. Cleary, and I. Iordachita, “Body-mounted robotic assistant for MRI-guided low back pain injection,” International journal of computer assisted radiology and surgery, vol. 15, no. 2, pp. 321-331, 2020.
[6] R. Monfaredi, K. Cleary, and K. Sharma, “MRI robots for needle-based interventions: systems and technology,” Annals of biomedical engineering, vol. 46, no. 10, pp. 1479-1497, 2018.
[7] A. Squires, J. N. Oshinski, N. M. Boulis, and Z. T. H. Tse, “Spinobot: an mri-guided needle positioning system for spinal cellular therapeutics,” Annals of biomedical engineering, vol. 46, no. 3, pp. 475-487, 2018.
[8] J. Esteban, W. Simson, S. R. Witzig, A. Rienmu\ddot{ }ller, S. Virga, B. Frisch, O. Zettinig, D. Sakara, Y.-M. Ryang, N. Navab et al., “Robotic ultrasound-guided facet joint insertion,” International journal of computer assisted radiology and surgery, vol. 13, no. 6, pp. 895-904, 2018.
[9] M. Tirindelli, M. Victorova, J. Esteban, S. T. Kim, D. Navarro-Alarcon, Y. P. Zheng, and N. Navab, “Force-ultrasound fusion: Bringing spine robotic-us to the next “level”,” IEEE Robotics and Automation Letters, vol. 5, no. 4, pp. 5661-5668, 2020.
[10] S. Schafer, S. Nithiananthan, D. Mirota, A. Uneri, J. Stayman, W. Zbijewski, C. Schmidgunst, G. Kleinszig, A. Khanna, and J. Siewerdsen, “Mobile c-arm cone-beam ct for guidance of spine surgery: Image quality, radiation dose, and integration with interventional guidance,” Medical physics, vol. 38, no. 8, pp. 4563-4574, 2011.
[11] S. Onogi, K. Morimoto, I. Sakuma, Y. Nakajima, T. Koyama, N. Sugano, Y. Tamura, S. Yonenobu, and Y. Momoi, “Development of the needle insertion robot for percutaneous vertebroplasty,” in International Conference on Medical Image Computing and Computer-Assisted Intervention. Springer, 2005, pp. 105-113.
[12] Z. Han, K. Yu, L. Hu, W. Li, H. Yang, M. Gan, N. Guo, B. Yang, H. Liu, and Y. Wang, “A targeting method for robot-assisted percutaneous needle placement under fluoroscopy guidance,” Computer Assisted Surgery, vol. 24, no. sup 1, pp. 44-52, 2019.
[13] G. Burstro\ddot{ }m, M. Balicki, A. Patriciu, S. Kyne, A. Popovic, R. Holthuizen, R. Homan, H. Skulason, O. Persson, E. Edstro\ddot{ }m et al., “Feasibility and accuracy of a robotic guidance system for navigated spine surgery in a hybrid operating room: a cadaver study,” Scientific reports, vol. 10, no. 1, pp. 1-9, 2020.
[14] C. Gao, A. Farvardin, R. B. Grupp, M. Bakhtiarinejad, L. Ma, M. Thies, M. Unberath, R. H. Taylor, and M. Armand, “Fiducial-free 2d/3d registration for robot-assisted femoroplasty,” IEEE Transactions on Medical Robotics and Bionics, vol. 2, no. 3, pp. 437-446, 2020.
[15] C. Gao, H. Phalen, S. Sefati, J. Ma, R. H. Taylor, M. Unberath, and M. Armand, “Fluoroscopic navigation for a surgical robotic system including a continuum manipulator,” IEEE Transactions on Biomedical Engineering, 2021.
[16] A. Fedorov, R. Beichel, J. Kalpathy-Cramer, J. Finet, J.-C. Fillion-Robin, S. Pujol, C. Bauer, D. Jennings, F. Fennessy, M. Sonka et al., “3d slicer as an image computing platform for the quantitative imaging network,” Magnetic resonance imaging, vol. 30, no. 9, pp. 1323-1341, 2012.
[17] R. B. Grupp, M. Armand, and R. H. Taylor, “Patch-based image similarity for intraoperative 2d/3d pelvis registration during periacetabular osteotomy,” in OR 2.0 Context-Aware Operating Theaters, Computer Assisted Robotic Endoscopy, Clinical Image-Based Procedures, and Skin Image Analysis. Springer, 2018, pp. 153-163.
[18] N. Hansen and A. Ostermeier, “Completely derandomized self-adaptation in evolution strategies,” Evolutionary computation, vol. 9, no. 2, pp. 159-195, 2001.
[19] M. Krc ̌ah, G. Sze\acute{ }kely, and R. Blanc, “Fully automatic and fast segmentation of the femur bone from 3d-ct images with no shape prior,” in 2011 IEEE international symposium on biomedical imaging: from nano to macro. IEEE, 2011, pp. 2087-2090.
[20] C. Kim, C. J. Moon, H. E. Choi, and Y. Park, “Retrodiscal approach of lumbar epidural block,” Annals of rehabilitation medicine, vol. 35, no. 3, p. 418, 2011.
[21] J. W. Park, H. S. Nam, S. K. Cho, H. J. Jung, B. J. Lee, and Y. Park, “Kambin's triangle approach of lumbar transforaminal epidural injection with spinal stenosis,” Annals of rehabilitation medicine, vol. 35, no. 6, p. 833, 2011.
[22] R. B. Grupp, M. Unberath, C. Gao, R. A. Hegeman, R. J. Murphy, C. P. Alexander, Y. Otake, B. A. McArthur, M. Armand, and R. H. Taylor, “Automatic annotation of hip anatomy in fluoroscopy for robust and efficient 2d/3d registration,” International journal of computer assisted radiology and surgery, vol. 15, no. 5, pp. 759-769, 2020.
[23] M. Unberath, J.-N. Zaech, C. Gao, B. Bier, F. Goldmann, S. C. Lee, J. Fotouhi, R. Taylor, M. Armand, and N. Navab, “Enabling machine learning in x-ray-based procedures via realistic simulation of image formation,” International journal of computer assisted radiology and surgery, vol. 14, no. 9, pp. 1517-1528, 2019.

### REFERENCES (EXAMPLE 2)

1. Hession W G, Stanczak J D, Davis K W, Choi J J. Epidural steroid injections. Semin Roentgenol. 2004; 39(1):7-23. doi: 10.1016/j.ro.2003.10.010.
2. Mathis J M. Epidural steroid injections. Neuroimaging Clin N Am. 2010; 20(2):193-202. doi: 10.1016/j.nic.2010.02.006.
3. Bicket M C, Gupta A, Brown CH, 4th, Cohen S P. Epidural injections for spinal pain: A systematic review and meta-analysis evaluating the “control” injections in randomized controlled trials. Anesthesiology. 2013; 119(4):907-931. doi: 10.1097/ALN.0b013e31829c2ddd.
4. Mandell J C, Czuczman G J, Gaviola G C, Ghazikhanian V, Cho C H. The lumbar neural foramen and transforaminal epidural steroid injections: An anatomic review with key safety considerations in planning the percutaneous approach. AJR Am J Roentgenol. 2017; 209(1):W26-W35. doi: 10.2214/AJR.16.17471.
5. Bui J, Bogduk N. A systematic review of the effectiveness of CT-guided, lumbar transforaminal injection of steroids. Pain Med. 2013; 14(12):1860-1865. doi: 10.1111/pme.12243.
6. White A H, Derby R, Wynne G. Epidural injections for the diagnosis and treatment of low-back pain. Spine. 1980; 5(1): 78-86. doi: 10.1097/00007632-198001000-00014.
7. Vad V B, Bhat A L, Lutz G E, Cammisa F. Transforaminal epidural steroid injections in lumbosacral radiculopathy: A prospective randomized study. Spine. 2002; 27(1):11-15. doi: 10.1097/00007632-200201010-00005.
8. Benny B V, Patel M Y. Predicting epidural steroid injections with laboratory markers and imaging techniques. Spine J. 2014; 14(10):2500-2508. doi: 10.1016/j.spinee.2014.04.003.
9. Lee M H, Yang K S, Kim Y H, JungDo H, Lim S J, Moon D E. Accuracy of live fluoroscopy to detect intravascular injection during lumbar transforaminal epidural injections. Korean J Pain. 2010; 23(1):18-23. doi: 10.3344/kjp.2010.23.1.18.
10. Smuck M, Fuller B J, Chiodo A, et al. Accuracy of intermittent fluoroscopy to detect intravascular injection during transforaminal epidural injections. Spine. 2008; 33(7):E205-E210. doi: 10.1097/BRS.0b013e31816960fe.
11. Faul F, Erdfelder E, Buchner A, Lang A-G. Statistical power analyses using G*Power 3.1: Tests for correlation and regression analyses. Behav Res Methods. 2009; 41(4):1149-1160. doi: 10. 3758/BRM.41.4.1149.
12. Bellingham G A, Peng P W H. A low-cost ultrasound phantom of the lumbosacral spine. Reg Anesth Pain Med. 2010; 35(3):290-293. doi: 10.1097/AAP.0b013e3181c75a76.
13. Park J W, Cheon M W, Lee M H. Phantom study of a new laser-etched needle for improving visibility during ultrasonography-guided lumbar medial branch access with novices. Ann Rehabil Med. 2016; 40(4):575-582. doi: 10.5535/arm.2016.40.4.575.
14. Fedorov A, Beichel R, Kalpathy-Cramer J, et al. 3D Slicer as an image computing platform for the quantitative imaging network. Magn Reson Imaging. 2012; 30(9):1323-1341. doi: 10.1016/j.mri.2012.05.001.
15. Hartley R, Zisserman A. Multiple View Geometry in Computer Vision. Cambridge: Cambridge University Press; 2004.
16. Gao C, Farvardin A, Grupp R B, et al. Fiducial-free 2D/3D registration for robot-assisted femoroplasty. IEEE Trans Med Robot bionics. 2020; 2(3):437-446. doi: 10.1109/tmrb.2020. 3012460.
17. Grupp R B, Armand M, Taylor RH. Patch-based image similarity for intraoperative 2D/3D pelvis registration during peri-acetabular osteotomy. Lect Notes Comput Sci. 2018; 11041 LNCS: 153-163. doi: 10.1007/978-3-030-01201-4_17.
18. Hansen N, Ostermeier A. Completely derandomized self-adaptation in evolution strategies. Evol Comput. 2001; 9(2): 159-195. doi: 10.1162/106365601750190398.
19. Grupp R B, Hegeman R A, Murphy R J, et al. Pose estimation of periacetabular osteotomy fragments with intraoperative X-Ray navigation. IEEE Trans Biomed Eng. 2020; 67(2):441-452. doi: 10.1109/TBME.2019.2915165.
20. Beyer L P, Michalik K, Niessen C, et al. Evaluation of a robotic assistance-system for percutaneous computed tomography-guided (CT-guided) facet joint injection: A phantom study. Med Sci Mon Int Med J Exp Clin Res. 2016; 22:3334-3339. doi: 10.12659/MSM.900686.
21. Li G, Patel N A, Melzer A, Sharma K, Iordachita I, Cleary K. MRI-guided lumbar spinal injections with body-mounted robotic system: cadaver studies. Minim Invasive Ther Allied Technol. 2020; 31:297-305. doi: 10.1080/13645706.2020.1799017.
22. Shoham M, Lieberman I H, Benzel E C, et al. Robotic assisted spinal surgery—from concept to clinical practice. Comput Aided Surg. 2007; 12:105-115. doi: 10.1080/10929080701243981.
23. D'Souza M, Gendreau J, Feng A, Kim L H, Ho A L, Veeravagu A. Robotic-assisted spine surgery: History, efficacy, cost, and future trends. Rob Surg Res Rev. 2019; 6:9-23. doi: 10.2147/rsrr.s190720.
24. Condon A. Robotics in Spine Surgery: 17 Notes for Surgeons, ASCs & Administrators: Becker's Spine Review; 2020. https://www.beckersspine.com/robotics/item/50394-robotics-in-spine-surgery-17-notes-for-surgeons-ascs-administrators.html.
25. Hoshide R, Feldman E, Taylor W. Cadaveric analysis of the Kambin's triangle. Cureus. 2016; 8(2):e475. doi: 10.7759/cureus.475.

While various embodiments of the present invention have been described above, it should be understood that these embodiments have been presented by way of example only, and not limitation. Thus, the breadth and scope of the present invention should not be limited by any of the above-described illustrative embodiments, or following examples, but should instead be defined only in accordance with the following claims and their equivalents.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. Moreover, features described in connection with one embodiment may be used in conjunction with other embodiments, even if not explicitly stated above.

1. An image-guided robotic spine injection system, comprising:
a spine injection robot comprising an end effector configured to hold an injection device, said spine injection robot being configured to be registered to an interoperative imaging system for real-time guidance of said injection device; and
a guidance system configured to communicate with said spine injection robot and said interoperative imaging system during an injection procedure,
wherein said guidance system comprises a preoperative injection plan for a planned injection procedure on a subject, said preoperative injection plan being based on preoperative imaging data of at least a portion of a subject's spine, said preoperative injection plan comprising a plurality of anatomical features identified as a corresponding plurality of preoperative registration markers,
wherein said guidance system is configured to receive interoperative imaging data from said interoperative imaging system of at least said portion of said subject's spine,
wherein said guidance system is further configured to receive as input from a user an indication of a plurality of anatomical features identified as a plurality of interoperative registration markers that correspond in a one-to-one relationship to each respective one of said plurality of preoperative registration markers,
wherein said guidance system is further configured to register said plurality of interoperative registration markers with said plurality of preoperative registration markers to transform said preoperative injection plan to an interoperative injection plan, and
wherein said guidance system is further configured to provide injection guidance instructions to said spine injection robot to perform autonomous injections into the spine of a subject by said injection device.

2. The system according to claim 1, wherein said plurality of anatomical features are at least a portion of each of a plurality of vertebrae of said subject, and
wherein said registering said plurality of interoperative registration markers with said plurality of preoperative registration markers accounts for relative movement of said subject's vertebrae in the interoperative imaging data compared to the preoperative imaging data.

3. The system according to claim 1, wherein said preoperative injection plan includes boundaries to prevent said injection device from damaging said subject's spinal cord or other nerves.

4. The system according to claim 1, further comprising a tracking system configured to communicate with said guidance system,
wherein said tracking system is arranged to be registered to and track said spine injection robot, said end effector of said spine injection robot, a needle and injection device when attached to said end effector, an imaging portion of said interoperative imaging system, and said plurality of vertebrae of said subject while in operation.

5. The system according to claim 4, wherein said tracking system provides closed-loop control of said spine injection robot based on tracking information from said tracking system.

6. The system according to claim 1, further comprising a preoperative planning module configured to receive preoperative imaging data of said at least said portion of said subject's spine,
wherein said preoperative planning module is further configured to receive a planned injection point and a planned destination point from a user and to display a corresponding calculated needle path to said user.

7. The system according to claim 1, further comprising said interoperative imaging system.

8. The system according to claim 7, wherein said preoperative imaging data is three-dimensional preoperative imaging data, and
wherein said interoperative imaging system is configured to provide a plurality of two-dimensional interoperative images from a plurality of different views.

9. A method for image guidance for robotic spine injection, comprising:
registering a spine injection robot to an interoperative imaging system for real-time guidance of an injection device coupled to said spine injection robot;
receiving preoperative imaging data of a subject's spine;
generating, based on said preoperative imaging data, a preoperative injection plan for a planned injection procedure on said subject, wherein said preoperative injection plan comprises a plurality of anatomical features identified as a corresponding plurality of preoperative registration markers;
receiving an indication of a plurality of anatomical features identified as a plurality of interoperative registration markers that correspond in a one-to-one relationship to each respective one of said plurality of preoperative registration markers;
registering said plurality of interoperative registration markers with said plurality of preoperative registration markers to transform said preoperative injection plan to an interoperative injection plan; and
providing injection guidance instructions to said spine injection robot to perform autonomous injections into said subject's spine by said injection device.

10. The method according to claim 9, wherein said plurality of anatomical features are at least a portion of each of a plurality of vertebrae of said subject, and
wherein the registering said plurality of interoperative registration markers with said plurality of preoperative registration markers accounts for relative movement of said subject's vertebrae in said interoperative imaging data compared to said preoperative imaging data.

11. The method according to claim 9, wherein said preoperative injection plan includes boundaries to prevent said injection device from damaging said subject's spinal cord or other nerves.

12. The method according to claim 9, wherein said preoperative imaging data comprises a planned injection point and a planned destination point from a user, the method further comprising displaying a corresponding calculated needle path to said user.

13. The method according to claim 9, wherein said preoperative imaging data comprises three-dimensional preoperative imaging data, and
wherein said interoperative imaging system is configured to provide a plurality of two-dimensional interoperative images from a plurality of different views.

14. The method of claim 9, wherein said spine injection robot comprises an end effector configured to hold said injection device.

15. The method according to claim 9, further comprising receiving tracking information from a tracking system,
wherein said tracking system is arranged to be registered to and track said spine injection robot, said end effector of said spine injection robot, a needle and injection device when attached to said end effector, an imaging portion of said interoperative imaging system, and a plurality of vertebrae of said subject while in operation.

16. The method according to claim 15, wherein said tracking system provides closed-loop control of said spine injection robot based on tracking information from said tracking system.

17. The method of claim 9, wherein the indication of said plurality of anatomical features is received as an input from a user.

18. A non-transitory computer-readable medium storing a set of instructions for image-guided robotic spine injection, which when executed by a processor, configure the processor to:
register a spine injection robot to an interoperative imaging system for real-time guidance of an injection device coupled to said spine injection robot;
receive preoperative imaging data of a subject's spine;
generate, based on said preoperative imaging data, a preoperative injection plan for a planned injection procedure on said subject, wherein said preoperative injection plan comprises a plurality of anatomical features identified as a corresponding plurality of preoperative registration markers;
receive an indication of a plurality of anatomical features identified as a plurality of interoperative registration markers that correspond in a one-to-one relationship to each respective one of said plurality of preoperative registration markers;
register said plurality of interoperative registration markers with said plurality of preoperative registration markers to transform said preoperative injection plan to an interoperative injection plan; and
provide injection guidance instructions to said spine injection robot to perform autonomous injections into said subject's spine by said injection device.

19. The non-transitory computer-readable medium according to claim 18, wherein said plurality of anatomical features are at least a portion of each of a plurality of vertebrae of said subject, and
wherein registering said plurality of interoperative registration markers with said plurality of preoperative registration markers accounts for relative movement of said subject's vertebrae in said interoperative imaging data compared to said preoperative imaging data.

20. The non-transitory computer-readable medium according to claim 18, wherein said preoperative imaging data comprises three-dimensional preoperative imaging data, and
wherein said interoperative imaging system is configured to provide a plurality of two-dimensional interoperative images from a plurality of different views.
